(19) United States
(12) Patent Application Publication
(10) Pub. No.: US 2024/0247228 A1
(43) Pub. Date: Jul. 25, 2024

(54) BIOLOGICALLY CONTAINED BACTERIA AND USES THEREOF

(71) Applicant: NOVOME BIOTECHNOLOGIES, INC., South San Francisco, CA (US)

(72) Inventors: Weston Robert Whitaker, Daly City, CA (US); William Cain DeLoache, Alameda, CA (US); Zachary Nicholas Russ, IV, San Bruno, CA (US); Elizabeth Joy Stanley Shepherd, Redwood City, CA (US); Lauren Popov, Redwood City, CA (US)

(21) Appl. No.: 17/618,130

(22) PCT Filed: Jun. 12, 2020
(86) PCT No.: PCT/US2020/037571

§ 371 Date: Aug. 24, 2022

Related U.S. Application Data
(60) Provisional application No. 62/861,181, filed on Jun. 13, 2019.

(51) Int. Cl. C12N 1/20; A61K 35/74; C07K 14/195; C12N 1/38; C12N 15/74

(52) U.S. Cl. CPC C12N 1/20; A61K 35/74; C07K 14/195; C12N 1/38; C12N 15/74

(57) ABSTRACT

The present disclosure provides biocontainment methods and mechanisms that prevent modified cells from escaping their intended environment(s) while enabling the survival and replication of the modified cells where intended. This is achieved by linking the viability of the modified cells to the presence of a control molecule that is exogenously supplied to define the location and time in which cells are capable of growing.

### CROSS REFERENCE TO RELATED APPLICATIONS

This application is a § 371 National Stage of International (PCT) Patent Application Serial No. PCT/US2020/037571, filed on Jun. 12, 2020, which claims the benefit of, and priority to, U.S. Provisional Patent Application No. 62/861,181, filed on Jun. 13, 2019, the disclosures of each of which are hereby incorporated by reference in their entirety for all purposes.

### GOVERNMENT RIGHTS

This invention was made with Government support under contract 1831185 awarded by the National Science Foundation. The Government has certain rights in the invention.

### SEQUENCE LISTING

This application contains a Sequence Listing that has been submitted in ASCII format via EFS-Web and is hereby incorporated by reference in its entirety. The ASCII copy, “NVM-003WOUS_SL.txt,” was created on Jul. 22, 2022 and is 832,196 bytes in size.

### BACKGROUND

Cell-based therapeutics are an emerging approach to complement the traditional small molecule and protein-based therapies in diseases where spatial and temporal specificity, logic and new activities are needed, but can only be developed by engineering whole cells. A challenge unique to cell-based therapeutics is controlling the replication of the therapeutic cells in a manner that does not interfere with the therapeutic function but can limit survival to a defined time and space. Biocontainment is a necessary feature of a genetically modified cellular therapeutic, whereby the therapeutic cell is modified to not be capable of reproducing outside of an intended location and/or duration. Therapeutics that persist beyond the intended treatment period or escape into the environment or other people represent a risk that must be addressed.

Introduced mutations that confer a fitness disadvantage, such as an auxotrophy that can only be complemented in the laboratory, offer an effective means of biocontainment. However, for many applications it will be necessary for the cellular therapeutics to be viable in patients, for instance to outcompete pathogenic microbes or to reach the abundance needed for efficacy. To enable controllable growth of cells in vivo, numerous strategies have been devised that make viability dependent on the presence of an easily controllable environmental signal, typically a small molecule. However, most biocontainment methods published to date make use of toxins which are induced as the means of killing cells in the presence of a control molecule. There are two disadvantages to this approach. First, the default state for these biocontained cells is to be alive, meaning that any cells that are not actively exposed to the control molecule when clearance is required will continue to persist. Complete clearance from a patient would require 100% of the therapeutic cells to come in contact with the appropriate concentration of control molecule, which is difficult to achieve in practice. This is particularly problematic in the context of a bacterial therapeutic, where rates of shedding are high and transmission from person-to-person is possible.

A second disadvantage to toxin-dependent biocontainment methods is the high frequency at which cells can escape, since any mutation that disables the toxin gene (e.g. nonsense mutations, transposon insertions, etc.) will break the biocontainment strategy. To reduce the escape rate, multiple copies of toxins can be encoded, thereby requiring multiple mutations for escape, which will be less frequent than a single mutation. Although this redundancy does successfully reduce the escape rate (Cai et al., (2015) PROC. NATL. ACAD. SCI. U. S. A. 112, 1803-1808; Chan et al., (2015) NAT. CHEM. BIOL. 12, 82-86; Gallagher et al., (2015) NUCLEIC ACIDS RES. 43, 1945-1954), mobile genetic elements are common in non-model organisms and, once induced to replicate, are capable of inserting into multiple locations with a high frequency. Any strategy in which loss-of-function mutations will break biocontainment, which includes all strategies that rely on toxins, suffers from this fundamental limitation.

As an alternative to using toxins, others have described strategies for linking a control molecule's presence to the expression of an essential gene, wherein in the absence of the control molecule, the essential gene is not produced, and the cells are no longer viable. This strategy avoids concerns over strain shedding, since the default state of the cells is death, and they must be actively supplied with the control molecule to remain alive.

Additionally, in contrast to toxins, mutations to the essential gene that render it non-functional will result in a loss of viability instead of escape from biocontainment. However, for many inducible viability strategies described to date, biocontainment is dependent on transcriptional repressors that block expression in the absence of the control molecule. Like the toxin-based strategies, repressor-based biocontainment can be easily subverted with a loss of function mutation that prevents the repressor from functioning and thus produces constitutive expression of the essential genes.

Accordingly, there is a need in the art for new biocontainment strategies that reduce or eliminate escape frequency.

### SUMMARY

The disclosure relates in part to the use of activators to activate essential gene expression for biocontainment of recombinant bacteria. In contrast to repressors, which, as discussed above, can be easily subverted with a loss of function mutation that prevents the repressor from functioning and thus produces constitutive expression of the essential gene, the most common mutations to an activator will result in no essential gene expression under any conditions, and thus will be less prone to escape.

One challenge, however, with the use of activators for biocontainment is that unlike repressors, in which including additional copies of the repressor offers some reduction in escape frequency, escape mutants for activators are dominant (only one of the copies would need to mutate to be constitutively active to subvert biocontainment). Therefore, providing additional copies of an activator provides no reduction in escape rate.

Disclosed herein are methods and compositions for biocontainment that take advantage of the rare rate of subverting activator-based biocontainment yet avoid the problems of dominant activator mutations that reduce the effectiveness of redundancy by redirecting small molecule sensing two component systems (TCSs) to control the expression of essential genes. Therapeutic strains of gut bacteria engineered in this way are capable of reproducing in the gut when patients ingest a control molecule sensed by the TCS but fail to reproduce in the patient when the control molecule is not ingested or in other environments lacking the control molecule. The disclosure provides compositions and methods for implementing this strategy in any organism and includes multiple working examples implementing porphyran dependent biocontainment in species of gut bacteria from the Bacteroides genus.

In one aspect, the disclosure relates to a genetically modified bacterium that includes a first activator that is activated by a control molecule, a first promoter that is activated by the first activator; and a first essential gene that is operably linked to the first promoter. In certain embodiments, the bacterium can include a second activator that is activated by the control molecule, a second promoter that is activated by the second activator, and a second essential gene that is operably linked to the second promoter. In certain embodiments, the first promoter is not activated by the second activator and the second promoter is not activated by the first activator.

In certain embodiments, the bacterium further comprises a third activator that is activated by the control molecule, a third promoter that is activated by the third activator, and a third essential gene that is operably linked to the third promoter. In certain embodiments, the third promoter is not activated by the first or second activator and the first or second promoter is not activated by the third activator.

In certain embodiments, the expression of the first, second, and/or third essential gene is dependent upon the presence of the control molecule. In certain embodiments, the growth and/or viability of the bacterium is dependent upon the presence of the control molecule. In certain embodiments, the control molecule is not regularly present in the human diet. In certain embodiments, the control molecule is a monosaccharide or a polysaccharide, for example, a marine polysaccharide or an antibiotic, or a derivative of any of the foregoing. In certain embodiments, the marine polysaccharide is porphyran or agarose, or a derivative of either of the foregoing. In certain embodiments, the antibiotic is anhydrotetracycline or derivative thereof.

In certain embodiments, the first, second, and/or third activator is a two-component system (TCS) protein comprising a sensor domain and a regulatory domain. In certain embodiments, the first, second, and/or third activator is a hybrid two-component system (HTCS) protein comprising a sensor domain and a regulatory domain.

In certain embodiments, the HTCS protein is a naturally occurring HTCS protein, or a functional fragment or variant thereof. For example, the naturally occurring HTCS protein can be a bacterial HTCS protein, such as a Bacteroides (e.g., Bacteroides ovatus, Bacteroides dorei, Bacteroides nordii, Bacteroides salyersiae, or Bacteroides uniformis) HTCS protein.

In certain embodiments, the HTCS protein is a chimeric HTCS protein, wherein the sensor domain is a sensor domain from a first naturally-occurring HTCS protein, or a functional fragment or variant thereof, and the regulatory domain is a regulatory domain from a second naturally-occurring HTCS protein, or a functional fragment or variant thereof.

In certain embodiments, the HTCS protein comprises an amino acid sequence having at least 80% identity to any one of SEQ ID NOs: 19, 23, 25, 38, 39, 42, 43, 51, 52, 53, 54, 59, or 64-71, or a functional fragment or variant thereof.

In certain embodiments, the bacterium comprises one or more transgenes encoding the first, second, and/or third activator.

In certain embodiments, the first, second, and/or third promoter comprises a nucleotide sequence having at least 80% identity to any one of SEQ ID NOs: 1, 2, 7, 8, 9, 10, 11, 12, 13, 45, 46, 62, 63, or 73, or a functional fragment or variant thereof, e.g., SEQ ID NO: 44.

In certain embodiments, the essential gene is selected from thymidylate synthase (Thy A), arginyl-tRNA synthetase (argS), cysteinyl-tRNA synthetase (cysS), penicillin tolerance protein (lytB) and peptide chain release factor (RF-2).

In certain embodiments, the first, second, and/or third activator and/or promoter is heterologous to the bacterium. In certain embodiments, the first, second, and/or third gene is not operably linked to the first, second, and/or third promoter, respectively, in a similar or otherwise identical bacterium that has not been modified.

In certain embodiments, culturing of the bacterium results in a bacterium that is capable of growth and/or viability in the absence of the control molecule at a frequency of less than 10⁻⁵, 10⁻⁶, 10⁻⁷, 10⁻⁸, or 10⁻⁹. In certain embodiments, following culture of the bacterium with the control molecule and subsequent removal of the control molecule from the culture, the half-life of the bacteria in the culture is less than a day. In certain embodiments, following administration of the bacterium and control molecule to a subject, the amount of bacteria in the subject decreases 10 fold within 2 days of removal or discontinuation of the control molecule from the subject.

In certain embodiments, the control molecule is a porphyran and the first and second activator are each an TCS or HTCS protein, and (i) the porphyran, when present, activates the first and second TCS or HTCS proteins, (ii) the first and second TCS or HTCS proteins, when activated, activate the first and second promoters, respectively, and (iii) the first and second promoters, when activated, direct expression of the first and second essential genes, respectively, thereby resulting in the growth and/or viability of the bacterium being dependent upon the presence of the porphyran. In certain embodiments, the bacterium is a commensal bacterium.

In certain embodiments, the bacterium further comprises one or more transgenes encoding a protein homologous to a starch binding protein such as SusC or SusD, e.g., SEQ ID NO: 20 or 21. In certain embodiments, the bacterium comprises one or more transgenes that increase its ability to utilize a privileged nutrient as carbon source, for example, a marine polysaccharide such as porphyran.

In certain embodiments, the bacterium further comprises one or more therapeutic transgenes. In certain embodiments, the therapeutic transgene is operably linked to a promoter, such as a non-native promoter (e.g., a phage-derived promoter). In certain embodiments, the promoter comprises the consensus sequence GTTAA(n)₄₋₇GTTAA(n)₃₄₋₃₈TA(n)₂TTTG. In certain embodiments, the promoter comprises SEQ ID NO: 48, SEQ ID NO: 49, or SEQ ID NO: 50. In certain embodiments, any of the transgenes are on a plasmid, on a bacterial artificial chromosome, and/or are genomically integrated.

In another aspect, the disclosure relates to a pharmaceutical composition comprising a bacterium as disclosed herein and a pharmaceutically acceptable excipient. In certain embodiments, the composition is formulated as a capsule, e.g., an enteric coated capsule, or a tablet. In certain embodiments, the composition further comprises the control molecule.

In another aspect, the disclosure relates to a method for reducing the growth and/or viability of a bacterium (e.g., a commensal bacterium) in the absence of a control molecule. The method includes genetically modifying the bacterium to comprise a first activator that is activated by the control molecule, a first promoter that is activated by the first activator, and a first essential gene that is operably linked to the first promoter. In certain embodiments, the method further includes genetically modifying the bacterium to comprise a second activator that is activated by the control molecule, a second promoter that is activated by the second activator, and a second essential gene that is operably linked to the second promoter.

In certain embodiments, the method further includes genetically modifying the bacterium to comprise a third activator that is activated by the control molecule, a third promoter that is activated by the third activator, and a third essential gene that is operably linked to the third promoter.

In another aspect, the disclosure relates to a protein (e.g., an isolated protein) comprising the amino acid sequence of any one of SEQ ID NOs: 39, 43, 53, 54, 59, or 64-71, or a functional fragment or variant thereof, or an amino acid sequence having at least 80%, at least 85%, at least 90%, at least 91%, at least 92%, at least 93%, at least 94%, at least 95%, at least 96%, at least 97%, at least 98%, or at least 99% identity to any one of SEQ ID NOs: 39, 43, 53, 54, 59, or 64-71, or a functional fragment or variant thereof. In additional aspects, the disclosure relates to a nucleic acid (e.g., an isolated nucleic acid) comprising a nucleotide sequence encoding the protein, an expression vector comprising the nucleic acid, a host cell (e.g., a bacterium) comprising the expression vector, and a pharmaceutical composition comprising the protein, nucleic acid, expression vector, or host cell.

In another aspect, the disclosure relates to a nucleic acid (e.g., an isolated nucleic acid) comprising the nucleotide sequence any one of SEQ ID NOs: 29, 30, 31, 34, 35, 36, 37, 40, 55, 56, 60, 61, or 72, or a functional fragment or variant thereof, or a nucleotide sequence having at least 80%, at least 85%, at least 90%, at least 91%, at least 92%, at least 93%, at least 94%, at least 95%, at least 96%, at least 97%, at least 98%, or at least 99% identity to any one of SEQ ID NOs: 29, 30, 31, 34, 35, 36, 37, 40, 55, 56, 60, 61, or 72, or a functional fragment or variant thereof. In additional aspects, the disclosure relates to an expression vector comprising the nucleic acid, a host cell (e.g., a bacterium) comprising the expression vector, and a pharmaceutical composition comprising the protein, nucleic acid, expression vector, or host cell.

In another aspect, the disclosure relates to a genetically modified bacterium that includes (i) an HTCS that is activated by porphyran comprising the amino acid of SEQ ID NO: 19, or a functional fragment or variant thereof, or an amino acid sequence having at least 80%, at least 85%, at least 90%, at least 91%, at least 92%, at least 93%, at least 94%, at least 95%, at least 96%, at least 97%, at least 98%, or at least 99% identity to SEQ ID NO: 19, or a functional fragment or variant thereof; (ii) a promoter that is activated by the HTCS comprising the nucleotide sequence of SEQ ID NO: 73, or a functional fragment or variant thereof, or a nucleotide sequence having at least 80%, at least 85%, at least 90%, at least 91%, at least 92%, at least 93%, at least 94%, at least 95%, at least 96%, at least 97%, at least 98%, or at least 99% identity to SEQ ID NO: 73, or a functional fragment or variant thereof; and (iii) an essential gene (e.g., an argS gene) that is operably linked to the promoter. In certain embodiments, the essential gene (e.g., the argS gene) is operably linked to a ribosome binding site (RBS) comprising the nucleotide sequence of SEQ ID NO: 47, or a functional fragment or variant thereof, or a nucleotide sequence having at least 80%, at least 85%, at least 90%, at least 91%, at least 92%, at least 93%, at least 94%, at least 95%, at least 96%, at least 97%, at least 98%, or at least 99% identity to SEQ ID NO: 47, or a functional fragment or variant thereof.

In another aspect, the disclosure relates to a genetically modified bacterium that includes (i) an HTCS that is activated by porphyran comprising the amino acid of SEQ ID NO: 59, or a functional fragment or variant thereof, or an amino acid sequence having at least 80%, at least 85%, at least 90%, at least 91%, at least 92%, at least 93%, at least 94%, at least 95%, at least 96%, at least 97%, at least 98%, or at least 99% identity to SEQ ID NO: 59, or a functional fragment or variant thereof; (ii) a promoter that is activated by the HTCS comprising the nucleotide sequence of SEQ ID NO: 45, or a functional fragment or variant thereof, or a nucleotide sequence having at least 80%, at least 85%, at least 90%, at least 91%, at least 92%, at least 93%, at least 94%, at least 95%, at least 96%, at least 97%, at least 98%, or at least 99% identity to SEQ ID NO: 45, or a functional fragment or variant thereof; and (iii) an essential gene (e.g., a lytB gene) that is operably linked to the promoter. In certain embodiments, the essential gene (e.g., the lytB gene) is operably linked to a ribosome binding site (RBS) comprising the nucleotide sequence of SEQ ID NO: 84, or a functional fragment or variant thereof, or a nucleotide sequence having at least 80%, at least 85%, at least 90%, at least 91%, at least 92%, at least 93%, at least 94%, at least 95%, at least 96%, at least 97%, at least 98%, or at least 99% identity to SEQ ID NO: 84, or a functional fragment or variant thereof.

In another aspect, the disclosure relates to a genetically modified bacterium that includes (i) a first HTCS that is activated by porphyran comprising the amino acid of SEQ ID NO: 19, or a functional fragment or variant thereof, or an amino acid sequence having at least 80%, at least 85%, at least 90%, at least 91%, at least 92%, at least 93%, at least 94%, at least 95%, at least 96%, at least 97%, at least 98%, or at least 99% identity to SEQ ID NO: 19, or a functional fragment or variant thereof; (ii) a first promoter that is activated by the first HTCS comprising the nucleotide sequence of SEQ ID NO: 73, or a functional fragment or variant thereof, or a nucleotide sequence having at least 80%, at least 85%, at least 90%, at least 91%, at least 92%, at least 93%, at least 94%, at least 95%, at least 96%, at least 97%, at least 98%, or at least 99% identity to SEQ ID NO: 73, or a functional fragment or variant thereof; (iii) a first essential gene (e.g., an argS gene) that is operably linked to the first promoter; (iv) a second HTCS that is activated by porphyran comprising the amino acid of SEQ ID NO: 59, or a functional fragment or variant thereof, or an amino acid sequence having at least 80%, at least 85%, at least 90%, at least 91%, at least 92%, at least 93%, at least 94%, at least 95%, at least 96%, at least 97%, at least 98%, or at least 99% identity to SEQ ID NO: 59, or a functional fragment or variant thereof; (v) a second promoter that is activated by the second HTCS comprising the nucleotide sequence of SEQ ID NO: 45, or a functional fragment or variant thereof, or a nucleotide sequence having at least 80%, at least 85%, at least 90%, at least 91%, at least 92%, at least 93%, at least 94%, at least 95%, at least 96%, at least 97%, at least 98%, or at least 99% identity to SEQ ID NO: 45, or a functional fragment or variant thereof; and (vi) a second essential gene (e.g., a lytB gene) that is operably linked to the second promoter. In certain embodiments, the first essential gene (e.g., the argS gene) is operably linked to a first ribosome binding site (RBS) comprising the nucleotide sequence of SEQ ID NO: 47, or a functional fragment or variant thereof, or a nucleotide sequence having at least 80%, at least 85%, at least 90%, at least 91%, at least 92%, at least 93%, at least 94%, at least 95%, at least 96%, at least 97%, at least 98%, or at least 99% identity to SEQ ID NO: 47, or a functional fragment or variant thereof. In certain embodiments, the second essential gene (e.g., the lytB gene) is operably linked to a second ribosome binding site (RBS) comprising the nucleotide sequence of SEQ ID NO: 84, or a functional fragment or variant thereof, or a nucleotide sequence having at least 80%, at least 85%, at least 90%, at least 91%, at least 92%, at least 93%, at least 94%, at least 95%, at least 96%, at least 97%, at least 98%, or at least 99% identity to SEQ ID NO: 84, or a functional fragment or variant thereof.

In another aspect, the disclosure relates to a method of colonizing the gut of a subject, the method comprising administering a bacterium or a pharmaceutical composition as described herein.

In another aspect, the disclosure relates to a method of treating a disease or disorder in a subject in need thereof, the method comprising administering a bacterium or a pharmaceutical composition as described herein to the subject. In certain embodiments, the method further includes administrating the control molecule to the subject. In certain embodiments, the control molecule is administered to the subject prior to, at the same time as, or after the bacterium. In certain embodiments, the bacterium or pharmaceutical composition is administered to the subject every 12 hours, 24 hours, day, 2 days, 3 days, 4 days, 5 days, 6 days, 7 days, week, 2 weeks, 3 weeks, 4 weeks, month, 2 months, 3 months, 4 months, 5 months, or 6 months. In certain embodiments, the time between consecutive administrations of the bacterium or pharmaceutical composition to the subject is about 1 day.

In certain embodiments, the subject is an animal, e.g., a human.

These and other aspects and features of the disclosure are described in the following detailed description and claims.

### DESCRIPTION OF THE DRAWINGS

The disclosure can be more completely understood with reference to the following drawings.

FIG. 1 shows comparisons of various biocontainment strategies, and the most likely mode of failure in which mutations subvert biocontainment.

FIG. 2 shows comparisons of redundancy implemented in various biocontainment strategies, and the most likely mode of failure in which mutations subvert biocontainment.

FIG. 3 depicts a series of bar graphs demonstrating identification of suitable control molecule promoter elements. FIG. 3A shows luciferase reporter induction of candidate porphyran-responsive promoters (SEQ ID NO: 1-10) in wildtype NB001 Bacteroides. Luminescence was measured and normalized by OD₆₀₀ₙₘ in the absence or presence of porphyran. FIG. 3B shows luciferase reporter induction of candidate agarose-responsive promoters (SEQ ID NO: 11, 12) in wildtype NB003. Luminescence was measured and normalized by OD₆₀₀ₙₘ in the absence or presence of agarose. FIG. 3C shows luciferase reporter induction of a putative tetracycline-responsive promoter (SEQ ID NO: 13) in wildtype NB004. Luminescence was measured and normalized by OD₆₀₀ₙₘ in the absence or presence of anhydrotetracycline.

FIG. 4 shows characterization of porphyran-inducible promoter P_por10. FIG. 4A depicts the plasmid map of a P_por10-driven luciferase construct (SEQ ID NO: 26). FIG. 4B depicts luminescence measured, normalized by OD₆₀₀ₙₘ, of wildtype NB001 transformed with the P_por10-driven luciferase plasmid grown in varying concentrations of porphyran.

FIG. 5 depicts a bar graph demonstrating that porphyran-inducible HTCS alone is not sufficient for porphyran-response. The P_por10-driven luciferase element was stimulated in NB004 containing the full porphyran polysaccharide utilization locus (PUL) or in NB004 containing only the hybrid two-component system (HTCS) of the porphyran PUL. Luminescence was measured and normalized by OD₆₀₀ₙₘ in the absence or presence of porphyran.

FIG. 6 depicts an in vitro growth assay showing porphyran-inducible regulation of essential gene thy A and porphyran-dependent biocontainment. FIG. 6A shows luminescence, normalized by OD₆₀₀ₙₘ, of P_por10-driven thy A-luciferase coupled to the degenerate RBS library (SEQ ID NO: 30) in media supplemented with porphyran. Each point is a clonal library member. FIG. 6B depicts the plasmid map of the P_por10-driven thy A expression construct (SEQ ID NO: 31). FIG. 6C shows the growth curves of wildtype (“wt”) strain NB001, thy A knockout (“KO”) strain NB023, and biocontained (“BC”) strain NB024. Strains were grown in standard BHIS media, media supplemented with thymidine, or media supplemented with porphyran. FIG. 6D shows the growth curves of biocontained strain NB024 in BHIS supplemented with 0.0% porphyran, 0.002% porphyran, 0.02% porphyran, or 0.2% porphyran.

FIG. 7 shows the plasmid map (corresponding to SEQ ID NO: 32) used for essential gene promoter replacement with the porphyran-inducible promoter.

FIG. 8 depicts growth curves demonstrating porphyran-inducible regulation of multiple essential genes. FIG. 8A depicts growth curves of wildtype strain NB075 carrying a porphyran PUL in porphyran free BHIS media and in media containing 0.2% porphyran. FIG. 8B depicts growth curves of thy A-deletion strain sWW090 carrying the porphyran-driven thyA gene in porphyran free media and in media containing 0.2% porphyran. FIG. 8C depicts growth curves of the strain sWW180 carrying the porphyran-driven argS gene in porphyran free media and in media containing 0.2% porphyran. FIG. 8D depicts growth curves of the strain sWW202 carrying the porphyran-driven cysS gene in porphyran free media and in media containing 0.2% porphyran. FIG. 8E depicts the growth of lytB-deletion strain sWW090 carrying the porphyran-driven lytB gene in porphyran free media and in media containing 0.2% porphyran. FIG. 8F depicts the growth of RF-2-deletion strain sWW206 carrying the porphyran-driven RF-2 gene in porphyran free media and in media containing 0.2% porphyran.

FIG. 9 depicts an in vitro chemostat growth assay comparing growth of a wildtype and a porphyran-dependent biocontained strain. BHIS media containing 0.5% porphyran was diluted out by replacing half the media with porphyran free BHIS every 8.7 hours. Colony Forming Units (CFUs) are monitored for wildtype strain sZR0103 (grey line) and biocontained strain sZR0250 (black line), and escapes of the biocontained strain that are capable of growing without porphyran (dashed black line).

FIG. 10 depicts line graphs demonstrating elimination of a wildtype and a porphyran-dependent strain from the gut of Sprague-Dawley rats following porphyran-withdrawal. Rats were gavaged on Day 0 with 10⁹ CFU of wildtype strain sWW808 containing only a porphyran-PUL, or porphyran-biocontained strain sWW805 and fed a diet supplemented with porphyran. After 3 days, half the rats from each group were switched to a diet lacking porphyran, while the other half remained on the porphyran-containing diet. CFU plating of the feces was used to determine eliminated strain abundance. FIG. 10A depicts the results of the in vivo experiment for wildtype strain sWW808. FIG. 10B depicts the results of the in vivo experiment for biocontained strain sWW805 and demonstrates rapid clearance of the biocontained strain following porphyran withdrawal. Shaded regions represent 95% confidence intervals.

FIG. 11 shows the plasmid map of the construct utilized for essential gene promoter replacement with the anhydrotetracycline-inducible promoter (SEQ ID NO: 37).

FIG. 12 depicts an in vitro growth assay comparing biocontainment of a wildtype, 1× biocontained porphyran-dependent strain, and 2× biocontained porphyran- and anhydrotetracycline-dependent strain. Wildtype strain NB075, porphyran-controlled cysS biocontained strain sWW202, and porphyran-controlled cysS/aTc-controlled argS double-biocontained strain sCG037 were monitored for growth in vitro. Strains were grown in rich media, media containing only porphyran, media containing only aTc, or media containing both porphyran and aTc. Both biocontained stains required nutrient supplementation in order to grow, but escape colonies were not observed in the absence of aTc and porphyran in only the 2× biocontained strain.

FIG. 13 depicts an in vitro growth assay performed in a chemostat comparing biocontainment of a wildtype and 2× biocontained porphyran- and anhydrotetracycline-dependent strain. Porphyran and aTc were removed from the media at day 1 through replacing 2.16 volumes of flask media with BHIS-only per day. At day 7, porphyran and aTc were reintroduced into the media to assess if viable cells were present, but no growth was detected.

FIG. 14 depicts the generation of chimeric HTCSs which can be used, for example, for double-biocontainment using a single control molecule. FIG. 14A depicts a schematic demonstrating the use of a chimeric HTCS to regulate multiple promoters with a single control molecule. FIG. 14B shows a plasmid map of construct pWW1267 utilized for expression of a chimeric HTCS with a porphyran-sensing domain from the NB001 porphyran-responsive HTCS and a regulatory domain from a Bacteroides nordiiHTCS (SEQ ID NO: 39). FIG. 14C is a bar graph depicting promoter-driven expression of luciferase in strain NB075 or NB075 transformed with a construct expressing one of three chimeric HTCS: HTCS-17106 (pWW1266), HTCS-10809 (pWW1265), or HTCS-17150) (pWW1267). Activity in the absence or presence of 0.2% porphyran in the media is shown with the light grey and black bars, respectively. Approximate fold change in activity in response to porphyran presence is shown above the bars for each chimeric HTCS.

FIG. 15 depicts the generation of an improved mutant chimeric HTCS for use in biocontainment. FIG. 15A depicts a schematic of an assay for measuring the activity of chimeric HTCSs, where luciferase is driven by a chimeric HTCS-associated promoter (SEQ ID NO: 45). FIG. 15B shows the resulting luciferase values for strains expressing mutant chimeric HTCSs when grown in the absence (x-axis) or presence (y-axis) of porphyran. Each dot represents a strain including a unique mutant, squares represent strains including replicates of the initially designed chimeric HTCS, and the triangle represents strain pWW1333 including an improved mutant chimeric HTCS. FIG. 15C further shows promoter activity in the presence of no HTCS (left), the initially designed chimeric HTCS (pWW1267; middle) and an improved mutant chimeric HTCS (pWW1333; right) in the absence (grey) or presence (black) of porphyran, as assessed by luminescence from the reporter plasmid (SEQ ID NO: 41).

FIG. 16 demonstrates that a wildtype porphyran-responsive HTCS (“WT HTCS”) and a chimeric HTCS (HTCS-17150v2, “chimeric HTCS”) each activate their associated promoters without crosstalk to the other promoter. Strains that were tested are identified on the X axis, and beneath each strain identifier is a schematic of the HTCS that is expressed in that strain, and the promoter used to drive luciferase expression in that strain. Grey and black bars represent luminescence in the absence or presence of porphyran.

FIG. 17 demonstrates growth, as shown by OD₆₀₀ₙₘ growth curves over time, in the presence (black lines) or absence (grey lines) of porphyran of strains that are non-biocontained (sWW180; upper left), biocontained with only a wildtype porphyran HTCS (NB075; upper right), biocontained with only a chimeric HTCS (sWW939; lower left), or double biocontained with a wildtype porphyran HTCS and a chimeric HTCS controlling different essential genes (sWW942; lower right). Shaded regions represents the 95% confidence intervals for each group (n=3).

FIG. 18 depicts the abundance of strains as measured by colony forming units (CFU) with single (sWW180; solid black line), double (sWW942; dashed black line), or no (NB075; solid grey line) biocontainment in a 100 ml chemostat of BHIS that initially contained 0.2% porphyran that was diluted out with fresh BHIS lacking porphyran. The limit of detection is indicated with a grey dashed line.

FIG. 19 demonstrates the abundance of a porphyran consuming, non-biocontained strain (NB144; left) and a biocontained strain (sZR0323; right), in mice harboring one of four different human microbiotas (donors A-D). Mice were gavaged with strains once, on day 1, and fed a diet containing porphyran for the first 4 weeks (solid lines) and then switched to a diet lacking porphyran (dashed lines). Shaded regions represents the 95% confidence intervals for each group (n=2).

### DETAILED DESCRIPTION

The present disclosure provides biocontainment methods and mechanisms that prevent modified cells from escaping their intended environment(s) while enabling the survival and replication of the modified cells where intended. This is achieved by linking the viability of the modified cells to the presence of a control molecule that is exogenously supplied to define the location and time in which cells are capable of growing. While the preferred embodiments of the present invention described herein enable controllable growth of modified bacterial cells in the gut, it will be obvious to those skilled in the art that such embodiments are provided by way of example only. Other embodiments could utilize different cell types (e.g. mammalian or yeast cells) or be tailored to different environments (e.g. the mouth, the skin, the soil, or industrial fermenters) without departing from the invention. In some cases, the biocontainment is spatial. In some cases, the biocontainment is positional. In some examples the biocontainment is temporal.

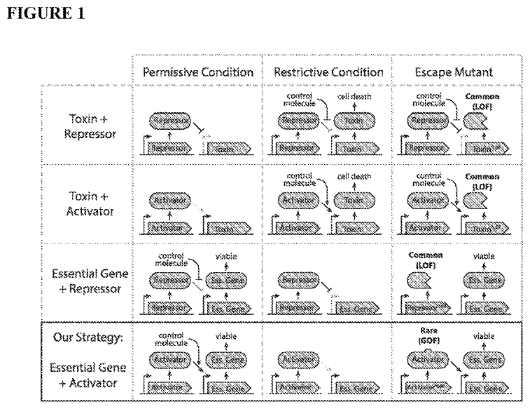

Alternative strategies for achieving control molecule dependent viability for biocontainment have been proposed previously and demonstrated in the laboratory, however none have been shown to be effective in vivo due to limitations related to high rates of strain escape, reliance on a control molecule not suitable for use in vivo, or severe decreases in fitness while implementing biocontainment that prevent colonization in even permissive conditions. FIG. 1 shows comparisons of various biocontainment strategies, and the most likely mode of failure in which mutations subvert biocontainment (right). Toxins and repressors can be disabled by common loss-of-function mutations. Activators can also be mutated to constitutively express a gene even in the absence of the control molecule, but this gain of function mutation is far less common.

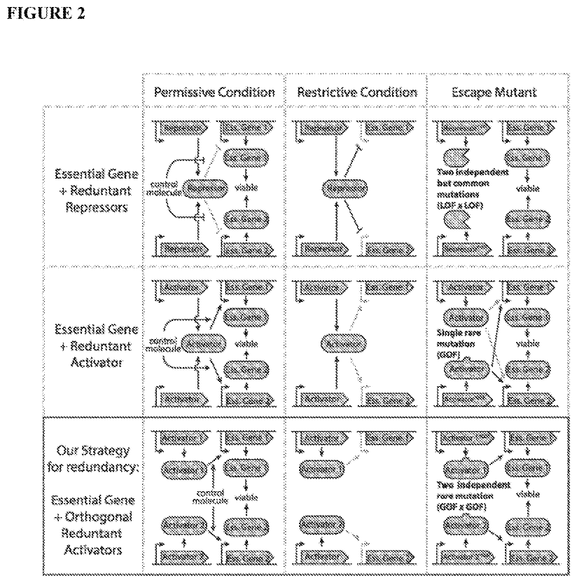

Escape from biocontainment that is based on activator driven expression of an essential gene requires a rare gain-of-function mutation that enables constitutive expression of the essential gene in the absence of the control molecule. One example of how this could be accomplished would be a mutation that renders the activator constitutively active. Though the reduced frequency of such a mutation is advantageous, when multiple essential genes are driven with the same control molecule as a means of adding redundancy, only one copy of the activator must be mutated in order to serve as a dominant mutation and activate all essential genes, thus reducing the ability to use redundancy to decrease the escape rate. FIG. 2 shows comparisons of redundancy implemented in various biocontainment strategies, and the most likely mode of failure in which mutations subvert biocontainment (right). Unlike repressors, mutations to subvert activators are likely to be dominant (middle row), and thus require orthogonal versions (bottom) to effectively add redundancy.

Accordingly, the disclosure relates, in part, to the discovery of biocontainment strategies using multiple activators that respond to the same molecule but target different promoters, such that a mutation rendering one activator constitutively active will not impact the other promoters. Identifying naturally-occurring activators of this type is extremely difficult, if not impossible. Accordingly, described herein are engineered two-component systems (TCSs) or hybrid two-component systems (HTCSs), which are usually activators (as opposed to repressors) and can be used to drive essential gene expression as a means of biocontainment. TCSs and HTCSs respond to many small molecules suitable for biocontainment in therapeutic or industrial applications. Such molecules include, but are not limited to, carbohydrates, metal ions, amino acids, phosphate, nitrate, pH, osmolarity, membrane stress and antibiotics.

The modular nature of TCSs and HTCSs allows for the engineering of multiple orthogonal versions that respond to the same molecule but activate different promoters. Canonical TCSs are composed of a sensor histidine kinase (HK), which responds to stimuli and activates a response regulator (RR), via a histidine-to-aspartic-acid phosphotransfer. When phosphorylated, the RR will activate or repress specific target promoters. HTCSs similarly regulate target promoters in a stimulus-dependent manner, but typically contain the sensor and DNA-binding regulatory domains on the same polypeptide. Most bacteria contain tens of TCSs or HTCSs that have low sequence identity, yet retain a high degree of structural similarity, with separate modular domains responsible for each signal transduction event. Due to this structural similarity, it is possible to generate a chimeric TCS or HTCS that redirects signal transduction from the sensor of one TCS or HTCS to the promoter of another.

Rewiring of signal transduction has been demonstrated in several academic publications (Lynch and Sonnenburg (2012) MOL. MICROBIOL. 85:478-491; Skerker et al., (2008) CELL 133: 1043-1054; Utsumi et al., (1989) SCIENCE 245:1246-1249; Whitaker et al., (2012) PROC. NATL. ACAD. SCI. U. S. A. 109:18090-18095), but the ability to engineer two orthogonal regulators that are induced simultaneously by the same molecule has not been shown. By engineering chimeric TCSs or HTCSs, multiple activators can respond to the same control molecule but not express the essential genes controlled by the other activators, preventing escape in the event that a mutation renders one TCS constitutively active. This approach provides a robust biocontainment system that can be implemented much more easily than existing options for redundant biocontainment, which necessitate widespread genome modifications that reduce organism fitness (Mandell et al., (2015) NATURE 518:55-60; Rovner et al., (2015) NATURE 518:89-93) or impose limitations on molecule choice (Lopez and Anderson, (2015) ACS SYNTH. BIOL. 4:1279-1286).

### I. Definitions

The term “heterologous” refers to genetic material that has been introduced to a cell wherein the genetic material is either not naturally present in the cell or is naturally present but with an altered sequence or genetic context compared to the introduced genetic material. The term “recombinant microorganism” refers to an organism which has been genetically modified to alter or remove native genetic material or to add heterologous genetic material. We refer primarily to bacterial cells, but it will be obvious to those skilled in the art that such embodiments are provided by way of example only. Other embodiments could utilize different cell types (e.g. mammalian or yeast cells) without departing from the invention.

The term “viability” refers to the potential for an organism to reproduce under certain environmental conditions. Cells that are viable in a given environmental condition are capable of reproducing in that environmental condition. Cells that are non-viable in a given environmental condition on are not capable of reproducing in that environmental condition.

The terms “biocontainment” or “biological containment” refer to a method of ensuring that the viability of an organism is restricted to a defined location and time.

The term “control molecule” refers to a molecule, typically referring to but not limited to an organic compound weighing less than 1500 Daltons, which can be used to control the viability of a biocontained recombinant microorganism.

The term “activator” refers to a gene, gene product, protein, or a portion thereof which increases the expression of a gene that it regulates under conditions of activation. When an activator is not functionally expressed (e.g. in the event of a loss-of-function mutation), the expression of the regulated gene is low, even under conditions of activation.

The term “repressor” refers to a gene, gene product, protein, or a portion thereof which reduces the expression of a gene that it regulates under conditions of repression. When a repressor is not functionally expressed (e.g. in the event of a loss-of-function mutation), the expression of the regulated gene is high, even under conditions of repression.

The term “toxin” refers to a gene whose product either directly or indirectly can result in the loss of viability under the condition of interest.

The term “essential gene” refers to a gene whose functional expression is necessary to maintain viability under the condition of interest.

The terms “two component system” (TCS) and “hybrid two component system” (HTCS) refer to a type of signal transduction pathway common in microorganisms, in which a sensor domain responds to an environmental signal (e.g. a molecule) and transduces the signal through conserved phosphotransfer domains which results in gene regulation, typically transcriptional regulation. There are two components in a canonical TCS, a histidine kinase and a response regulator. In a HTCS, the phosphotransfer domains are not canonically arranged, and domains associated with the histidine kinase and response regulator can be contained in a single protein. Herein, most principles apply to both TCS and HTCS and the terms TCS and HTCS are used interchangeably herein unless otherwise indicated.

The term “escape frequency” refers to the frequency at which biocontainment fails in a particular group of cells. For instance, a biocontainment implementation “with an escape frequency of 10⁻⁵” will produce a population of cells in which one cell in 10⁵ will be found to be viable outside of the conditions to which they have been restricted (e.g. when the control molecule is not present). Escape from biocontainment is typically the result of mutations that have disrupted the biocontainment mechanism.

The term “homology” or “sequence identity” used herein, may refer to a nucleotide-to-nucleotide or amino acid-to-amino acid correspondence of two polynucleotide or polypeptide sequences respectively. Sequence identity may be measured by any suitable alignment algorithm; for example using the BLAST algorithm (see e.g., the BLAST alignment tool available at blast.ncbi.nlm.nih.gov/Blast.cgi). Other alignment algorithms may also be used to measure the percent sequence identity between multiple polynucleotide or polypeptide sequences.

The term “therapeutic transgene” refers to a heterologous gene or DNA sequence which is capable of imparting a therapeutic benefit.

The term “diagnostic transgene” refers to a heterologous gene or DNA sequence which can be used to diagnose a condition or disease state.

As used herein, the term “functional fragment” of a biological entity (e.g., a gene, protein (e.g., an HTCS), promoter, or ribosome binding site) refers to a fragment of the full-length biological entity that retains, for example, at least 10%, at least 20%, at least 30%, at least 40%, at least 50%, at least 60%, at least 70%, at least 80%, at least 90%, or 100% of the biological activity of the corresponding full-length biologically entity.

### II. Two-Component Systems

The disclosure relates, in part, to a genetically modified bacterium that includes an activator, a promoter, and an essential gene operably linked to the promoter which can serve, in certain embodiments, to achieve biocontainment. The activator, promoter, and essential gene of the genetically modified bacterium can comprise a two-component system or hybrid two-component system (TCS or HTCS). When the bacterium is exposed to a control molecule, the control molecule binds to and activates the activator, which activates the promoter, causing the essential gene to be expressed. Accordingly, in certain embodiments, growth and/or viability of the bacterium is dependent upon the presence of the control molecule, which regulates expression of the essential gene.

In certain embodiments, the activator is a single polypeptide. In certain embodiments, the activator comprises two or more polypeptides. For example, an activator can be a single polypeptide that can both sense (e.g., bind to) a control molecule and activate a promoter. In certain embodiments, the activator comprises two polypeptides, one polypeptide that can sense (e.g., bind to) a control molecule and one polypeptide that can activate a promoter.

To avoid biocontainment escape, which can occur when the TCS or HTCS mutates to become constitutively active (e.g., by point mutation) or through alternative mechanism (e.g., by transposon insertions into the promoter, genomic rearrangements upstream of the essential gene, etc.), multiple TCSs or HTCSs can be used. In particular, incorporating different activator/promoter pairs that do not cross-activate provides redundancy and reduces the escape rate.

Accordingly, in certain embodiments, the bacterium can also include a second activator that is activated by the same control molecule or a different control molecule, a second promoter that is activated by the second activator, and a second essential gene that is operably linked to the second promoter. In certain embodiments, the first promoter is not activated by the second activator and the second promoter is not activated by the first activator.

In certain embodiments, the bacterium further comprises a third activator that is activated by the same control molecule or a different molecule, a third promoter that is activated by the third activator, and a third essential gene that is operably linked to the third promoter. In certain embodiments, the third promoter is not activated by the first or second activator and the third promoter is not activated by the first or second activator. In certain embodiments, the three activators are activated by three different control molecules, in certain embodiments, the three activators are activated by two different control molecules (i.e., one control molecule activates two of the activators, but not the third), and in certain embodiments, the three activators are activated by the same control molecule.

In certain embodiments, the bacterium comprises one or more transgenes encoding the first, second, and/or third activator.

In certain embodiments, the first, second, and/or third activator is a two-component system or hybrid two-component system (TCS or HTCS) protein comprising a sensor domain and a regulatory domain. In certain embodiments, the sensor domain binds to a control molecule, and the regulatory domain activates the promoter of the essential gene. In certain embodiments, the first, second, and/or third activator is a hybrid two-component system (HTCS) protein comprising a sensor domain and a regulatory domain.

In certain embodiments, the regulatory domain comprises an AraC family helix-turn-helix motif (see, e.g., Religa et al. (2007) PNAS 102(22):9272-7).

The TCS or HTCS protein can be a naturally occurring TCS or HTCS protein, or a functional fragment or variant thereof. For example, the naturally occurring TCS or HTCS protein can be a bacterial TCS or HTCS protein, such as a Bacteroides (e.g., Bacteroides ovatus, Bacteroides dorei, Bacteroides nordii, Bacteroides salyersiae, or Bacteroides uniformis) HTCS protein.

In certain embodiments, the TCS or HTCS protein is a chimeric TCS or HTCS protein, wherein the sensor domain is a sensor domain from a first naturally-occurring TCS or HTCS protein, or a functional fragment or variant thereof, and the regulatory domain is a regulatory domain from a second naturally-occurring TCS or HTCS protein, or a functional fragment or variant thereof.

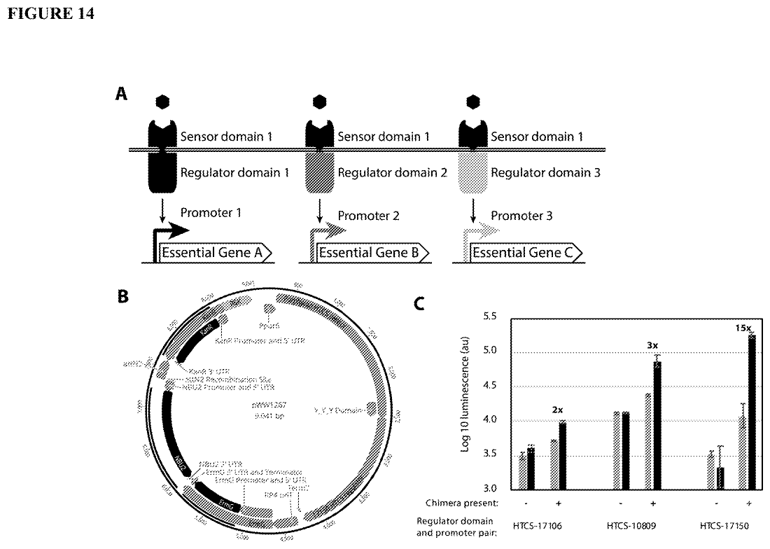

In one embodiment of the chimeric HTCS protein, the sensor of one HTCS is linked to the DNA-binding region of a second HTCS (see, e.g., FIG. 14A). This can be done by replacing the sensor domain of a second HTCS with the sensor domain of the first HTCS such that the chimeric HTCS senses the control molecule but targets a different promoter than the first, as described in more detail in Example 6.

To create a chimeric TCS, the sensor domain of one TCS (e.g., a naturally-occurring TCS) can be used in conjunction with the regulatory domain of a second TCS (e.g., a naturally-occurring TCS). Unlike an HTCS protein, in the chimeric TCS, the sensor domain and the regulatory domain are on separate polypeptides, and therefore only one of the two polypeptides (either the histidine kinase or the response regulator) will be a “chimeric” protein in the traditional sense. However, a similar system can be designed, for example, by engineering a bacterium that comprises the sensor domain of a first TCS with the regulatory domain of a first TCS and the regulatory domain of a second TCS, whereby the sensor domain of the first TCS activates the regulatory domain of both the first and second TCS.

As it is important to consider that the newly designed promoter only responds to the chimeric activation molecule and not to molecules produced by or commonly encountered by the host or to other HTCS or other regulators native to the host, the TCS or HTCS should contain regulatory domains either absent or rarely found in the biocontained strain.

In certain embodiments, the HTCS protein comprises the amino acid sequence of SEQ ID NO: 19, 23, 25, 38, 39, 42, 43, 51, 52, 53, 54, 59, or 64-71, or a functional fragment or variant thereof, or an amino acid sequence having at least 80%, at least 85%, at least 90%, at least 91%, at least 92%, at least 93%, at least 94%, at least 95%, at least 96%, at least 97%, at least 98%, or at least 99% identity to any one of SEQ ID NOs: 19, 23, 25, 38, 39, 42, 43, 51, 52, 53, 54, 59, or 64-71, or a functional fragment or variant thereof.

The sensor domain is typically about half of the total protein sequence and the regulatory domain is the remaining half of the protein. The regulatory domain may, e.g., comprise a DNA-binding domain, e.g., a helix-loop-helix domain, that recognizes a promoter sequence. In certain embodiments, the HTCS protein of SEQ ID NO: 19 comprises a sensor domain from about amino acid 1 to about amino acid 751, a regulatory domain from about amino acid 752 to about amino acid 1323, with a DNA-binding domain from about amino acid 1233 to about amino acid 1313. In certain embodiments, the HTCS protein of SEQ ID NO: 23 comprises a sensor domain from about amino acid 1 to about amino acid 787, a regulatory domain from about amino acid 788 to about amino acid 1368, with a DNA-binding domain from about amino acid 1279 to about amino acid 1359. In certain embodiments, the HTCS protein of SEQ ID NO: 25 comprises a sensor domain from about amino acid 1 to about amino acid 248, a regulatory domain from about amino acid 249 to about amino acid 772, with a DNA-binding domain from about amino acid 699 to about amino acid 772. In certain embodiments, the HTCS protein of SEQ ID NO: 38 comprises a sensor domain from about amino acid 1 to about amino acid 774, a regulatory domain from about amino acid 775 to about amino acid 1349, with a DNA-binding domain from about amino acid 1261 to about amino acid 1341. In certain embodiments, the HTCS protein of SEQ ID NO: 39 comprises a sensor domain from about amino acid 1 to about amino acid 751, a regulatory domain from about amino acid 752 to about amino acid 1326, with a DNA-binding domain from about amino acid 1238 to about amino acid 1318. In certain embodiments, the HTCS protein of SEQ ID NO: 42 comprises a sensor domain from about amino acid 1 to about amino acid 768, a regulatory domain from about amino acid 769 to about amino acid 1336, with a DNA-binding domain from about amino acid 1249 to about amino acid 1329. In certain embodiments, the HTCS protein of SEQ ID NO: 43 comprises a sensor domain from about amino acid 1 to about amino acid 751, a regulatory domain from about amino acid 752 to about amino acid 1319, with a DNA-binding domain from about amino acid 1232 to about amino acid 1312. In certain embodiments, the HTCS protein of SEQ ID NO: 51 comprises a sensor domain from about amino acid 1 to about amino acid 775, and a regulatory domain from about amino acid 776 to about amino acid 1349, with a DNA-binding domain from about amino acid 1259 to about amino acid 1339. In certain embodiments, the HTCS protein of SEQ ID NO: 52 comprises a sensor domain from about amino acid 1 to about amino acid 760, a regulatory domain from about amino acid 761 to about amino acid 1311, with a DNA-binding domain from about amino acid 1226 to about amino acid 1306. In certain embodiments, the HTCS protein of SEQ ID NO: 53 comprises a sensor domain from about amino acid 1 to about amino acid 751, a regulatory domain from about amino acid 752 to about amino acid 1325, with a DNA-binding domain from about amino acid 1235 to about amino acid 1315. In certain embodiments, the HTCS protein of SEQ ID NO: 54 comprises a sensor domain from about amino acid 1 to about amino acid 751. a regulatory domain from about amino acid 752 to about amino acid 1302, with a DNA-binding domain from about amino acid 1217 to about amino acid 1297. In certain embodiments, the HTCS protein of SEQ ID NO: 59 comprises a sensor domain from about amino acid 1 to about amino acid 751, a regulatory domain from about amino acid 752 to about amino acid 1326, with a DNA-binding domain from about amino acid 1238 to about amino acid 1318. In certain embodiments, the HTCS protein of SEQ ID NO: 64 comprises a sensor domain from about amino acid 1 to about amino acid 751, a regulatory domain from about amino acid 752 to about amino acid 1326, with a DNA-binding domain from about amino acid 1238 to about amino acid 1318. In certain embodiments, the HTCS protein of SEQ ID NO: 65 comprises a sensor domain from about amino acid 1 to about amino acid 751, a regulatory domain from about amino acid 752 to about amino acid 1326, with a DNA-binding domain from about amino acid 1238 to about amino acid 1318. In certain embodiments, the HTCS protein of SEQ ID NO: 66 comprises a sensor domain from about amino acid 1 to about amino acid 751, a regulatory domain from about amino acid 752 to about amino acid 1326, with a DNA-binding domain from about amino acid 1238 to about amino acid 1318. In certain embodiments, the HTCS protein of SEQ ID NO: 67 comprises a sensor domain from about amino acid 1 to about amino acid 751, a regulatory domain from about amino acid 752 to about amino acid 1326, with a DNA-binding domain from about amino acid 1238 to about amino acid 1318. In certain embodiments, the HTCS protein of SEQ ID NO: 68 comprises a sensor domain from about amino acid 1 to about amino acid 751, a regulatory domain from about amino acid 752 to about amino acid 1326, with a DNA-binding domain from about amino acid 1238 to about amino acid 1318. In certain embodiments, the HTCS protein of SEQ ID NO: 69 comprises a sensor domain from about amino acid 1 to about amino acid 751, a regulatory domain from about amino acid 752 to about amino acid 1326, with a DNA-binding domain from about amino acid 1238 to about amino acid 1318. In certain embodiments, the HTCS protein of SEQ ID NO: 70 comprises a sensor domain from about amino acid 1 to about amino acid 751, a regulatory domain from about amino acid 752 to about amino acid 1326, with a DNA-binding domain from about amino acid 1238 to about amino acid 1318. In certain embodiments, the HTCS protein of SEQ ID NO: 71 comprises a sensor domain from about amino acid 1 to about amino acid 751, a regulatory domain from about amino acid 752 to about amino acid 1326, with a DNA-binding domain from about amino acid 1238 to about amino acid 1318.

Accordingly, in certain embodiments, a contemplated HTCS protein comprises a sensor domain comprising an amino acid sequence comprising amino acids 1-751 of SEQ ID NO: 19, 1-787 of SEQ ID NO: 23, 1-248 of SEQ ID NO: 25, 1-774 of SEQ ID NO: 38, 1-751 of SEQ ID NO: 39, 1-768 of SEQ ID NO: 42, 1-751 of SEQ ID NO: 43, 1-775 of SEQ ID NO: 51, 1-760 of SEQ ID NO: 52, 1-751 of SEQ ID NO: 53, 1-751 of SEQ ID NO: 54, 1-751 of SEQ ID NO: 59, 1-751 of SEQ ID NO: 64, 1-751 of SEQ ID NO: 65, 1-751 of SEQ ID NO: 66, 1-751 of SEQ ID NO: 67, 1-751 of SEQ ID NO: 68, 1-751 of SEQ ID NO: 69, 1-751 of SEQ ID NO: 70, or 1-751 of SEQ ID NO: 71, or a functional fragment or variant thereof, or an amino acid sequence having at least 80%, at least 85%, at least 90%, at least 91%, at least 92%, at least 93%, at least 94%, at least 95%, at least 96%, at least 97%, at least 98%, or at least 99% identity to amino acids 1-751 of SEQ ID NO: 19, 1-787 of SEQ ID NO: 23, 1-248 of SEQ ID NO: 25, 1-774 of SEQ ID NO: 38, 1-751 of SEQ ID NO: 39, 1-768 of SEQ ID NO: 42, 1-751 of SEQ ID NO: 43, 1-775 of SEQ ID NO: 51, 1-760 of SEQ ID NO: 52, 1-751 of SEQ ID NO: 53, 1-751 of SEQ ID NO: 54, 1-751 of SEQ ID NO: 59, 1-751 of SEQ ID NO: 64, 1-751 of SEQ ID NO: 65, 1-751 of SEQ ID NO: 66, 1-751 of SEQ ID NO: 67, 1-751 of SEQ ID NO: 68, 1-751 of SEQ ID NO: 69, 1-751 of SEQ ID NO: 70, or 1-751 of SEQ ID NO: 71.

In certain embodiments, a contemplated HTCS protein comprises a regulatory domain comprising an amino acid sequence comprising amino acids 752-1323 or 1233-1313 of SEQ ID NO: 19, 788-1368 or 1279-1359 of SEQ ID NO: 23, 249-772 or 699-772 of SEQ ID NO: 25, 775-1349 or 1261-1341 of SEQ ID NO: 38, 752-1326 or 1238-1318 of SEQ ID NO: 39, 769-1336 or 1249-1329 of SEQ ID NO: 42, 752-1319 or 1232-1312 of SEQ ID NO: 43, 776-1349 or 1259-1339 of SEQ ID NO: 51, 761-1311 or 1226-1306 of SEQ ID NO: 52, 752-1325 or 1235-1315 of SEQ ID NO: 53, 752-1302 or 1217-1297 of SEQ ID NO: 54, 752-1326 or 1238-1318 of SEQ ID NO: 59, 752-1326 or 1238-1318 of SEQ ID NO: 64, 752-1326 or 1238-1318 of SEQ ID NO: 65, 752-1326 or 1238-1318 of SEQ ID NO: 66, 752-1326 or 1238-1318 of SEQ ID NO: 67, 752-1326 or 1238-1318 of SEQ ID NO: 68, 752-1326 or 1238-1318 of SEQ ID NO: 69, 752-1326 or 1238-1318 of SEQ ID NO: 70, or 752-1326 or 1238-1318 of SEQ ID NO: 71, or a functional fragment or variant thereof, or an amino acid sequence having at least 80%, at least 85%, at least 90%, at least 91%, at least 92%, at least 93%, at least 94%, at least 95%, at least 96%, at least 97%, at least 98%, or at least 99% identity to amino acids 752-1323 or 1233-1313 of SEQ ID NO: 19, 788-1368 or 1279-1359 of SEQ ID NO: 23, 249-772 or 699-772 of SEQ ID NO: 25, 775-1349 or 1261-1341 of SEQ ID NO: 38, 752-1326 or 1238-1318 of SEQ ID NO: 39, 769-1336 or 1249-1329 of SEQ ID NO: 42, 752-1319 or 1232-1312 of SEQ ID NO: 43, 776-1349 or 1259-1339 of SEQ ID NO: 51, 761-1311 or 1226-1306 of SEQ ID NO: 52, 752-1325 or 1235-1315 of SEQ ID NO: 53, 752-1302 or 1217-1297 of SEQ ID NO: 54, 752-1326 or 1238-1318 of SEQ ID NO: 59, 752-1326 or 1238-1318 of SEQ ID NO: 64, 752-1326 or 1238-1318 of SEQ ID NO: 65, 752-1326 or 1238-1318 of SEQ ID NO: 66, 752-1326 or 1238-1318 of SEQ ID NO: 67, 752-1326 or 1238-1318 of SEQ ID NO: 68, 752-1326 or 1238-1318 of SEQ ID NO: 69, 752-1326 or 1238-1318 of SEQ ID NO: 70, or 752-1326 or 1238-1318 of SEQ ID NO: 71. In certain embodiments, a contemplated HTCS protein comprises (i) a sensor domain comprising an amino acid sequence comprising amino acids 1-751 of SEQ ID NO: 19, 1-787 of SEQ ID NO: 23, 1-248 of SEQ ID NO: 25, 1-774 of SEQ ID NO: 38, 1-751 of SEQ ID NO: 39, 1-768 of SEQ ID NO: 42, 1-751 of SEQ ID NO: 43, 1-775 of SEQ ID NO: 51, 1-760 of SEQ ID NO: 52, 1-751 of SEQ ID NO: 53, 1-751 of SEQ ID NO: 54, 1-751 of SEQ ID NO: 59, 1-751 of SEQ ID NO: 64, 1-751 of SEQ ID NO: 65, 1-751 of SEQ ID NO: 66, 1-751 of SEQ ID NO: 67, 1-751 of SEQ ID NO: 68, 1-751 of SEQ ID NO: 69, 1-751 of SEQ ID NO: 70, or 1-751 of SEQ ID NO: 71, or a functional fragment or variant thereof, or an amino acid sequence having at least 80%, at least 85%, at least 90%, at least 91%, at least 92%, at least 93%, at least 94%, at least 95%, at least 96%, at least 97%, at least 98%, or at least 99% identity to amino acids 1-751 of SEQ ID NO: 19, 1-787 of SEQ ID NO: 23, 1-248 of SEQ ID NO: 25, 1-774 of SEQ ID NO: 38, 1-751 of SEQ ID NO: 39, 1-768 of SEQ ID NO: 42, 1-751 of SEQ ID NO: 43, 1-775 of SEQ ID NO: 51, 1-760 of SEQ ID NO: 52, 1-751 of SEQ ID NO: 53, 1-751 of SEQ ID NO: 54, 1-751 of SEQ ID NO: 59, 1-751 of SEQ ID NO: 64, 1-751 of SEQ ID NO: 65, 1-751 of SEQ ID NO: 66, 1-751 of SEQ ID NO: 67, 1-751 of SEQ ID NO: 68, 1-751 of SEQ ID NO: 69, 1-751 of SEQ ID NO: 70, or 1-751 of SEQ ID NO: 71: and (ii) a regulatory domain comprising an amino acid sequence comprising amino acids 752-1323 or 1233-1313 of SEQ ID NO: 19, 788-1368 or 1279-1359 of SEQ ID NO: 23, 249-772 or 699-772 of SEQ ID NO: 25, 775-1349 or 1261-1341 of SEQ ID NO: 38, 752-1326 or 1238-1318 of SEQ ID NO: 39, 769-1336 or 1249-1329 of SEQ ID NO: 42, 752-1319 or 1232-1312 of SEQ ID NO: 43, 776-1349 or 1259-1339 of SEQ ID NO: 51, 761-1311 or 1226-1306 of SEQ ID NO: 52, 752-1325 or 1235-1315 of SEQ ID NO: 53, 752-1302 or 1217-1297 of SEQ ID NO: 54, 752-1326 or 1238-1318 of SEQ ID NO: 59, 752-1326 or 1238-1318 of SEQ ID NO: 64, 752-1326 or 1238-1318 of SEQ ID NO: 65, 752-1326 or 1238-1318 of SEQ ID NO: 66, 752-1326 or 1238-1318 of SEQ ID NO: 67, 752-1326 or 1238-1318 of SEQ ID NO: 68, 752-1326 or 1238-1318 of SEQ ID NO: 69, 752-1326 or 1238-1318 of SEQ ID NO: 70, or 752-1326 or 1238-1318 of SEQ ID NO: 71, or a functional fragment or variant thereof, or an amino acid sequence having at least 80%, at least 85%, at least 90%, at least 91%, at least 92%, at least 93%, at least 94%, at least 95%, at least 96%, at least 97%, at least 98%, or at least 99% identity to amino acids 752-1323 or 1233-1313 of SEQ ID NO: 19, 788-1368 or 1279-1359 of SEQ ID NO: 23, 249-772 or 699-772 of SEQ ID NO: 25, 775-1349 or 1261-1341 of SEQ ID NO: 38, 752-1326 or 1238-1318 of SEQ ID NO: 39, 769-1336 or 1249-1329 of SEQ ID NO: 42, 752-1319 or 1232-1312 of SEQ ID NO: 43, 776-1349 or 1259-1339 of SEQ ID NO: 51, 761-1311 or 1226-1306 of SEQ ID NO: 52, 752-1325 or 1235-1315 of SEQ ID NO: 53, 752-1302 or 1217-1297 of SEQ ID NO: 54, 752-1326 or 1238-1318 of SEQ ID NO: 59, 752-1326 or 1238-1318 of SEQ ID NO: 64, 752-1326 or 1238-1318 of SEQ ID NO: 65, 752-1326 or 1238-1318 of SEQ ID NO: 66, 752-1326 or 1238-1318 of SEQ ID NO: 67, 752-1326 or 1238-1318 of SEQ ID NO: 68, 752-1326 or 1238-1318 of SEQ ID NO: 69, 752-1326 or 1238-1318 of SEQ ID NO: 70, or 752-1326 or 1238-1318 of SEQ ID NO: 71.

A first domain (e.g., a sensor domain) and a second domain (e.g., a regulatory domain) in a contemplated protein (e.g., an HTCS protein) may be coupled by a linker. The linker may be a cleavable linker or a non-cleavable linker. Optionally or in addition, the linker may be a flexible linker or an inflexible linker. The linker should be a length sufficiently long to allow the first and second domains to be linked without steric hindrance from one another and sufficiently short to retain the intended activity of the protein. The linker preferably is sufficiently hydrophilic to avoid or minimize instability of the protein. The linker preferably is sufficiently hydrophilic to avoid or minimize insolubility of the protein. The linker should be sufficiently stable in vivo (e.g., it is not cleaved by enzymes, etc.) to permit the fusion protein to be operative in vivo.

The linker may be from about 1 angstroms (Å) to about 150 Å in length, or from about 1 Å to about 120 Å in length, or from about 5 Å to about 110 Å in length, or from about 10 Å to about 100 Å in length. The linker may be greater than about 2, 3, 4, 5, 6, 7, 8, 9, 10, 11, 12, 13, 14, 15, 16, 17, 18, 19, 20, 25, 27, 30 or greater angstroms in length and/or less than about 110, 100, 90, 85, 80, 75, 70, 65, 60, 55, 50, 45, 43, 42, 41, 40, 39, 38, 37, 36, 35, 34, 33, 32, 31, or fewer Å in length. Furthermore, the linker may be about 5, 10, 15, 20, 25, 30, 35, 40, 45, 50, 55, 60, 65, 70, 75, 80, 85, 90, 95, 100, 110, and 120 Å in length.

In certain embodiments, the linker comprises a polypeptide linker. When a linker is employed, the linker may comprise hydrophilic amino acid residues, such as Gln, Ser, Gly, Glu, Pro, His and Arg. In certain embodiments, the linker is a peptide containing 1-25 amino acid residues, 1-20 amino acid residues, 2-15 amino acid residues, 3-10 amino acid residues, 3-7 amino acid residues, 4-25 amino acid residues, 4-20 amino acid residues, 4-15 amino acid residues, 4-10 amino acid residues, 5-25 amino acid residues, 5-20 amino acid residues, 5-15 amino acid residues, 5-10 amino acid residues, or 1, 2, 3, 4, 5, 6, 7, 8, 9, or 10 amino acid residues. Exemplary linkers include glycine and serine-rich linkers, e.g., (GlyGlyPro)ₙ, or (GlyGlyGlyGlySer)ₙ, where n is 1-5. In certain embodiments, the linker is (Gly₄Ser)₂. Additional exemplary linker sequences are disclosed, e.g., in George et al. (2003) PROTEIN ENGINEERING 15:871-879, and U.S. Pat. Nos. 5,482,858 and 5,525,491. In certain embodiments, the linker is derived from a naturally occurring protein, e.g., a naturally occurring HTCS protein. In certain embodiments, the linker comprises NPPF (SEQ ID NO: 78), KAPW (SEQ ID NO: 79), APPF (SEQ ID NO: 80), LPPW (SEQ ID NO: 81), or KPPF (SEQ ID NO: 82). In certain embodiments, the linker comprises 4 or more amino acid residues, of which 2 or more are proline. For example, In certain embodiments, the linker comprises X₁PPX₄ (SEQ ID NO: 83), wherein X₁ and X₄ are any amino acid.

Use of an TCS or HTCS reduces the escape rate of a bacterial strain. In certain embodiments, culturing of the bacterium results in a bacterium that is capable of growth and/or viability in the absence of the control molecule at a frequency of less than 10⁻⁵, 10⁻⁶, 10⁻⁷, 10⁻⁸, or 10⁻⁹. In certain embodiments, following culture of the bacterium with the control molecule and subsequent removal of the control molecule from the culture, the bacteria is viable in the culture for less than 3 days, less than 2 days, less than a day, or less than 12 hours. In certain embodiments, following culture of the bacterium with the control molecule and subsequent removal of the control molecule from the culture, the bacteria is capable of dividing less than 10 times, 9 times, 8 times, 7 times, 6 times, 5 times, 4 times, 3 time, twice or once.

In certain embodiments, following administration of the bacterium and control molecule to a subject, e.g., a human subject, the amount of bacteria in the subject decreases at least about 10-fold, 5-fold, or 2 fold within 2 days of removal or discontinuation of the control molecule from the subject. The amount of bacteria in the subject can be measured by any means known in the art, for example, by quantitative PCR (e.g., of the therapeutic gene), or by plating a sample on plates containing the control molecule as the sole carbon source and counting CFUs.

In certain embodiments, the first, second, and/or third promoter comprises a the nucleotide sequence of any one of SEQ ID NOs: 1-13, 44-46, 62, 63, or 73, or a functional fragment or variant thereof, or nucleotide sequence having at least 80%, at least 85%, at least 90%, at least 95%, at least 96%, at least 97%, at least 98%, or at least 99% identity to any one of SEQ ID NOs: 1-13, 44-46, 62, 63, or 73, or a functional fragment or variant thereof. In certain embodiments, the first, second, and/or third promoter comprises the nucleotide sequence of SEQ ID NO: 1, 2, 7, 8, 9, 10, 11, 12, 13, 45, 46, 62, 63, or 73, or a functional fragment or variant thereof (e.g., SEQ ID NO: 44), or a nucleotide sequence having at least 80%, at least 85%, at least 90%, at least 95%, at least 96%, at least 97%, at least 98%, or at least 99% identity to any one of SEQ ID NOs: 1, 2, 7, 8, 9, 10, 11, 12, 13, 45, 46, 62, 63, or 73, or a functional fragment or variant thereof (e.g., SEQ ID NO: 44). SEQ ID NO: 44, called Ppor10s6v7, is a minimal porphyran-responsive promoter, which is a truncated form of SEQ ID NO: 8 comprising mutations which can, in certain embodiments, improve activity.

In certain embodiments, the first, second, and/or third activator and/or promoter is heterologous to the bacterium. In certain embodiments, the first, second, and/or third gene is not operably linked to the first, second, and/or third promoter, respectively, in a similar or otherwise identical bacterium that has not been modified.

In addition to implementing a system in which essential genes are directly transcriptionally controlled by TCSs or HTCSs as described above, it will be recognized by those skilled in the art that this system may also be implemented with TCSs or HTCSs indirectly regulating essential gene function. For instance, the TCSs or HTCSs may control expression of one or more different activators which then drive expression of the essential gene. Those skilled in the art will also recognize alternatives to transcriptional regulation as a means of functionally linking TCS or HTCS activity to essential gene function. For instance, the biocontainment strategy described here can also be implemented by controlling essential gene translation, maturation, post-translational modification or localization. For instance, the TCS or HTCS may drive expression of RNA molecules that alter translation initiation, chaperones that ensure proper protein folding. proteases that mediate post-translational processing, or a variety of other factors that may be used alone or in combination to indirectly control essential gene function. Those skilled in the art will also recognize that the principle of TCS or HTCS regulation of essential genes can be applied to redundant gene pairs that on their own are not essential but when both deleted together result in a loss of viability. In this case, the TCS or HTCS can be linked to the function of both genes as a means of controlling viability or one of the redundant genes can simply be deleted to ensure that the other is essential on its own.

In certain embodiments, biocontainment is implemented with a carbohydrate-control biocontainment strategy, whereby the ability of the recombinant microbe to grow on carbohydrates found in the gut is limited and a control molecule is supplied. Limiting the ability of the recombinant microbe to grow on carbohydrates found in the gut can be achieved by knocking out a native polysaccharide utilization locus (PULs). PULs can be identified by searching for putative operons that contain SusC and SusD homologs (see, e.g., Xu et al. (2003). SYMBIOSIS 299, 2074-2077, which identified at least 12 putative PULs in B. thetaiotaomicron: BTO139-BT0146, BT0188- BT0196, BT0752-BT0758, BT1278-BT1287, BT1617-BT1622, BT1871-BT1877, BT2189-BT2198, BT2457-BT2463, BT3517-BT3532, BT3748-BT3754, BT4629-BT4636 and BT4722-BT4730). PULs can be deleted in full or in part using established methods (Koropatkin et al. (2008) STRUCTURE 16, 1105-1115). Deletion of a single PUL or multiple PULs can be used to partially or fully eliminate viability in the gut. The deletion of multiple PULs can be performed in series using established methods (Koropatkin et al., supra). A heterologous PUL can then be introduced to impart the ability to grow on a carbohydrate not commonly found in the gut. Though a large number of carbohydrate-PUL pairs may be capable of at least partially restoring viability, the ideal carbohydrate would be one that is not degraded by other gut microbes, such as the porphyran PUL described above. Transfer of the porphyran PUL can be performed as described in Examples below.

### IV. Essential Genes

An essential gene is a gene whose functional expression is necessary to maintain viability under the condition of interest. In certain embodiments, the essential gene is selected from thymidylate synthase (ThyA), arginyl-tRNA synthetase (argS), cysteinyl-tRNA synthetase (cysS), penicillin tolerance protein (lytB) and peptide chain release factor (RF-2). Other exemplary essential genes include those listed in TABLE 1. Table 1 provides predicted essential genes for B. thetaiotaomicron (Goodman et al. (2009) CELL HOST MICROBE 6(3):279-289.) Essential genes for other bacteria are known in the art, or can be identified as genes having 80% or more sequence identity to those listed in TABLE 1 (e.g., genes that are orthologous to those listed in TABLE 1).

TABLE 1

| | Gene length | |
|---|---|---|
| GeneID | (bp) | Annotation |
| BT0004 | 668 | hypothetical protein |
| BT0048 | 476 | hypothetical protein |
| BT0119 | 443 | conserved hypothetical protein |
| BT0130 | 815 | putative oxidoreductase |
| BT0205 | 1925 | glutamine-dependent NAD+ synthetase |
| BT0251 | 743 | dolichol-phosphate mannosyltransferase |
| BT0286 | 446 | hypothetical protein |
| BT0287 | 425 | putative biopolymer transmembrane protein |
| BT0307 | 1646 | phosphofructokinase |
| BT0319 | 371 | conserved hypothetical protein |
| BT0328 | 2042 | conserved hypothetical protein |
| BT0337 | 482 | hypothetical protein |
| BT0375 | 689 | integrase |
| BT0402 | 509 | hypothetical protein |
| BT0422 | 1940 | threonyl-tRNA synthetase |
| BT0423 | 557 | translation initiation factor IF-3 |
| BT0437 | 1190 | N-acylglucosamine 2-epimerase |
| BT0475 | 593 | putative phosphoheptose isomerase |
| BT0546 | 725 | hypothetical protein |
| BT0547 | 1232 | aspartate aminotransferase |
| BT0552 | 1340 | glutamate synthase, small subunit |
| BT0560 | 1229 | outer membrane efflux protein |
| BT0577 | 1766 | LysM-repeat proteins and domains |
| BT0589 | 1856 | putative inner membrane protein translocase |
| | | com . . . |
| BT0590 | 1613 | CTP synthase (UTP-ammonia ligase) |
| BT0595 | 956 | integrase |
| BT0624 | 824 | 4-diphosphocytidyl-2-C-methyl-D-erythritol |
| | | kinase |
| BT0625 | 1553 | DNA helicase |
| BT0626 | 2462 | phenylalanyl-tRNA synthetase beta chain |
| BT0688 | 668 | cAMP-binding domain (catabolite gene activator) |
| | | transcriptional regulator |
| BT0698 | 821 | 3-methyl-2-oxobutanoate hydroxymethyltrans- |
| | | ferase |
| BT0743 | 2339 | penicillin-binding protein 1A (PBP-1a) |
| BT0745 | 752 | 3-deoxy-manno-octulosonate cytidylyltrans- |
| | | ferase |
| BT0748 | 917 | ribose-phosphate pyrophosphokinase |
| BT0758 | 572 | acetyltransferase |
| BT0789 | 887 | malonyl CoA-acyl carrier protein transacylase |
| BT0795 | 620 | similar to DNA-binding protein |
| BT0806 | 3488 | isoleucyl-tRNA synthetase |
| BT0834 | 1097 | putative permease |
| BT0850 | 524 | putative transcriptional regulator |
| BT0872 | 1763 | aspartyl-tRNA synthetase |
| BT0883 | 830 | hypothetical protein |
| BT0888 | 776 | AMP nucleosidase |
| BT0889 | 1019 | similar to DNA polymerase III, delta subunit |
| BT0890 | 470 | putative DNA-binding protein |
| BT0894 | 2000 | DNA ligase |
| BT0895 | 893 | dihydrodipicolinate synthase |
| BT0899 | 2576 | DNA gyrase subunit A |
| BT0914 | 959 | recognition particle-docking protein FtsY |
| BT0920 | 1019 | putative O-sialoglycoprotein endopeptidase |
| BT0922 | 881 | putative lipoprotein |
| BT0928 | 677 | two-component system response regulator |
| BT0929 | 1493 | prolyl-tRNA synthetase |
| BT0934 | 290 | hypothetical protein |
| BT0947 | 587 | integrase |
| BT0972 | 809 | putative oxidoreductase |
| BT0976 | 1253 | putative transport protein |
| BT1021 | 851 | arabinosidase |
| BT1053 | 608 | RNA polymerase ECF-type sigma factor |
| BT1055 | 626 | pyruvate formate-lyase activating enzyme |
| BT1066 | 569 | conserved hypothetical protein |
| BT1124 | 926 | putative integrase |
| BT1215 | 1220 | ABC transporter, permease protein |
| BT1263 | 527 | putative protease I |
| BT1274 | 638 | L-fuculose-1-phosphate aldolase |
| BT1311 | 860 | RNA polymerase sigma factor rpoD (Sigma-A) |
| BT1317 | 602 | riboflavin synthase alpha chain |
| BT1325 | 1739 | glutaminyl-tRNA synthetase |
| BT1335 | 1475 | folylpolyglutamate synthase |
| BT1362 | 1202 | flavoprotein |
| BT1363 | 833 | DNA Pol III Epsilon Chain |
| BT1364 | 1124 | DNA polymerase III, beta chain |
| BT1368 | 995 | UDP-N-acetylenolpyruvoylglucosamine reductase |
| BT1369 | 848 | conserved hypothetical protein |
| BT1384 | 419 | hypothetical protein |
| BT1475 | 1157 | ABC transporter, permease protein |
| BT1484 | 1571 | conserved hypothetical protein with a |
| | | conserved domain |
| BT1495 | 605 | siderophore (surfactin) biosynthesis |
| | | regulatory protein |
| BT1500 | 1574 | Ribonuclease G |
| BT1541 | 581 | putative transmembrane protein |
| BT1593 | 1280 | putative cell-cycle protein |
| BT1595 | 2168 | transcription termination factor rho |
| BT1601 | 1322 | putative signal recognition protein |
| BT1610 | 1859 | DNA polymerase III subunit gamma/tau |
| BT1637 | 626 | conserved hypothetical protein |
| BT1669 | 1019 | phenylalanyl-tRNA synthetase alpha chain |
| BT1672 | 1259 | phosphoglycerate kinase |
| BT1691 | 1004 | fructose-bisphosphate aldolase |
| BT1700 | 1199 | hypothetical protein |
| BT1732 | 653 | amino acid exporter, putative |
| BT1829 | 1637 | 60 kDa chaperonin (groEL) |
| BT1840 | 1364 | histidyl-tRNA synthetase |
| BT1873 | 983 | endo-arabinase |
| BT1880 | 1142 | tetraacyldisaccharide 4′-kinase |
| BT1942 | 440 | hypothetical protein |
| BT1964 | 608 | hypothetical protein |
| BT1975 | 1508 | tRNA nucleotidyltransferase |
| BT2003 | 857 | putative membrane peptidase |
| BT2005 | 1304 | UDP-N-acetylglucosamine 1- |
| | | carboxyvinyltransferase |
| BT2007 | 695 | putative glycoprotease |
| BT2009 | 614 | guanylate kinase (GMP kinase) |
| BT2011 | 608 | putative nicotinate-nucleotide |
| | | adenylyltransferase |
| BT2016 | 1136 | dTDP-glucose 4,6-dehydratase |
| BT2017 | 875 | glucose-1-phosphate thymidylyltransferase |
| BT2047 | 794 | thymidylate synthase |
| BT2060 | 689 | cytidylate kinase |
| BT2061 | 869 | penicillin tolerance protein LytB |
| BT2122 | 1730 | lysyl-tRNA synthetase |
| BT2123 | 1037 | glycerol-3-phosphate dehydrogenase |
| BT2124 | 1337 | glucose-6-phosphate isomerase |
| BT2133 | 2708 | hypothetical protein |
| BT2143 | 1412 | chromosomal replication initiator |
| | | protein dnaA |
| BT2151 | 1028 | glycosyltransferase |
| BT2152 | 854 | putative acetyltransferase |
| BT2153 | 1331 | putative Fe—S oxidoreductases |
| BT2165 | 701 | two-component system response regulator |
| BT2177 | 608 | putative membrane protein |
| BT2184 | 494 | RNA polymerase ECF-type sigma factor |
| BT2192 | 1454 | putative lipoprotein |
| BT2206 | 809 | Zinc ABC transporter, permease |
| BT2230 | 3803 | DNA polymerase III alpha subunit |
| BT2231 | 686 | phosphatidylserine decarboxylase |
| BT2232 | 707 | CDP-diacylglycerol--serineO-phosphatidyl- |
| | | transferase |
| BT2238 | 746 | putative biotin--(acetyl-CoA carboxylase) |
| | | synthetase |
| BT2242 | 710 | uridylate kinase |
| BT2249 | 560 | ribosome recycling factor (ribosome |
| | | releasing factor) |
| BT2250 | 932 | putative GTPase |
| BT2282 | 572 | hypothetical protein |
| BT2293 | 1007 | conserved protein found in conjugate transposon |
| BT2372 | 947 | transcriptional regulator |
| BT2416 | 1214 | GTP cyclohydrolase II |
| BT2417 | 1850 | putative permease |
| BT2517 | 1841 | GcpE, 1-hydroxy-2-methyl-2-(E)-butenyl 4- |
| | | diphosphate synthase |
| BT2521 | 1484 | RNA polymerase sigma-54 |
| BT2525 | 1358 | cephalosporin-C deacetylase |
| BT2543 | 962 | riboflavin biosynthesis protein ribF, putative |
| | | riboflavin kinase |
| BT2548 | 995 | leucine aminopeptidase precursor |
| BT2584 | 824 | hypothetical protein |
| BT2595 | 929 | conserved protein found in conjugate transposon |
| BT2596 | 1298 | conserved protein found in conjugate transposon |
| BT2645 | 1082 | conserved hypothetical protein |
| BT2701 | 992 | DNA-directed RNA polymerase alpha chain |
| BT2702 | 605 | 30S ribosomal protein S4 |
| BT2704 | 380 | 30S ribosomal protein S13 |
| BT2707 | 1343 | preprotein translocase SecY subunit |
| BT2708 | 446 | 50S ribosomal protein L15 |
| BT2710 | 518 | 30S ribosomal protein S5 |
| BT2712 | 569 | 50S ribosomal protein L6 |
| BT2715 | 557 | 50S ribosomal protein L5 |
| BT2721 | 731 | 30S ribosomal protein S3 |
| BT2724 | 824 | 50S ribosomal protein L2 |
| BT2726 | 626 | 50S ribosomal protein L4 |
| BT2727 | 617 | 50S ribosomal protein L3 |
| BT2729 | 2117 | elongation factor G |
| BT2733 | 4283 | DNA-directed RNA polymerase beta′ chain |
| BT2734 | 3812 | DNA-directed RNA polymerase beta chain |
| BT2736 | 518 | ribosomal protein L10 |
| BT2737 | 698 | 50S ribosomal protein L1 |
| BT2739 | 542 | transcription anti-termination protein |
| BT2740 | 1184 | elongation factor Tu |
| BT2747 | 1223 | 3-deoxy-D-manno-octulosonic-acid transferase |
| BT2748 | 1514 | glutamyl-tRNA synthetase |
| BT2752 | 2456 | primosomal protein N′ (replication factor Y) |
| BT2754 | 980 | hypothetical protein |
| BT2761 | 638 | conserved hypothetical protein, similar to |
| | | O-methyltransferase |
| BT2765 | 551 | hypothetical protein |
| BT2796 | 2531 | hypothetical protein |
| BT2829 | 1793 | arginyl-tRNA synthetase |
| BT2838 | 1226 | putative lipoprotein releasing system |
| | | transmembrane permease |
| BT2883 | 584 | phosphoribosylglycinamide formyltransferase |
| BT2917 | 674 | conserved hypothetical protein |
| BT2925 | 602 | hypothetical protein |
| BT2985 | 446 | DNA repair protein |
| BT3031 | 626 | hypothetical protein |
| BT3033 | 1877 | DNA topoisomerase IV subunit B |
| BT3053 | 704 | putative cytochrome B subunit |
| BT3055 | 755 | succinate dehydrogenase iron-sulfur protein |
| BT3089 | 1490 | putative outer membrane protein, probably |
| | | involved in nutrient binding |
| BT3118 | 845 | prolipoprotein diacylglyceryl transferase |
| BT3126 | 2834 | leucyl-tRNA synthetase |
| BT3135 | 1232 | integrase |
| BT3212 | 794 | putative bacitracin resistance protein |
| BT3214 | 1058 | S-adenosylmethionine:tRNA ribosyltransferase- |
| | | isomerase |
| BT3219 | 1292 | S-adenosylmethionine synthetase |
| BT3230 | 1292 | tyrosyl-tRNA synthetase |
| BT3283 | 413 | conserved hypothetical protein with |
| | | conserved domain |
| BT3284 | 1232 | putative spore maturation protein A/B |
| BT3286 | 767 | hypothetical protein |
| BT3287 | 1448 | polysaccharide biosynthesis protein |
| BT3319 | 1481 | signal peptidase I |
| BT3351 | 1481 | cysteinyl-tRNA synthetase |
| BT3358 | 1262 | 3-oxoacyl-[acyl-carrier-protein] |
| | | synthase II |
| BT3386 | 1835 | ABC transporter, ATP-binding protein |
| BT3403 | 1319 | putative nitrogen utilization substance protein |
| BT3404 | 3122 | translation initiation factor IF-2 |
| BT3406 | 1454 | ABC transporter permease |
| BT3407 | 752 | ABC transporter ATP-binding protein |
| BT3408 | 1343 | conserved hypothetical protein, putative ABC |
| | | transporter permease component |
| BT3409 | 1211 | aminotransferase, putative cysteine desulfurase |
| BT3429 | 1961 | DNA gyrase subunit B |
| BT3435 | 317 | hypothetical protein |
| BT3438 | 422 | hypothetical protein |
| BT3444 | 1307 | cell division protein FtsZ |
| BT3445 | 1451 | cell division protein FtsA |
| BT3446 | 746 | cell division protein FtsQ |
| BT3447 | 1403 | UDP-N-acetylmuramate--alanine ligase |
| BT3448 | 1118 | UDP-N-acetylglucosamine--N-acetylmuramyl- |
| | | (pentapeptide) pyrophosphoryl- |
| | | undecaprenol N- |
| | | acetylglucosamine transferase |
| BT3449 | 1316 | rod shape-determining protein rodA |
| BT3450 | 1241 | UDP-N-acetylmuramoylalanine--D-glutamate |
| | | ligase |
| BT3451 | 1268 | phospho-N-acetylmuramoyl-pentapeptide- |
| | | transferase |
| BT3452 | 1448 | UDP-N-acetylmuramoylalanyl-D-glutamate--2,6- |
| | | diaminopimelate ligase |
| BT3453 | 2126 | penicillin-binding protein |
| BT3455 | 914 | S-adenosyl-methyltransferase mraW |
| BT3499 | 734 | conserved hypothetical protein |
| BT3532 | 1133 | aldose 1-epimerase precursor |
| BT3534 | 680 | hypothetical protein |
| BT3552 | 959 | peptide chain release factor RF-2 |
| BT3573 | 347 | hypothetical protein |
| BT3579 | 2552 | topoisomerase IV subunit A |
| BT3611 | 1541 | glycyl-tRNA synthetase |
| BT3636 | 1007 | aspartate-semialdehyde dehydrogenase |
| BT3638 | 2135 | Na+/H+ anti-porter |
| BT3639 | 716 | ThiF family protein, putative dinucleotide- |
| | | utilizing enzyme involved in molybdopterin |
| | | and thiamine biosynthesis |
| BT3640 | 656 | lipoprotein releasing system ATP-binding protein |
| BT3644 | 1298 | UDP-N-acetylmuramoylalanyl-D-glutamyl-2,6- |
| | | diaminopimelate-D-alanyl-D-alanyl ligase |
| BT3646 | 863 | dihydropteroate synthase |
| BT3647 | 686 | putative transmembrane protein |
| BT3652 | 788 | hypothetical protein |
| BT3653 | 566 | hypothetical protein |
| BT3692 | 1019 | phosphate acetyltransferase |
| BT3697 | 764 | UDP-2,3-diacylglucosamine hydrolase |
| BT3711 | 650 | hypothetical protein |
| BT3713 | 974 | D-alanine--D-alanine ligase |
| BT3714 | 1148 | Phospholipid/glycerol acyltransferase |
| BT3722 | 842 | glutamate racemase |
| BT3725 | 2657 | putative outer membrane protein |
| BT3726 | 734 | undecaprenyl pyrophosphate synthetase |
| BT3728 | 1019 | riboflavin biosynthesis protein ribD |
| BT3771 | 746 | 3-oxoacyl-[acyl-carrier protein] reductase |
| BT3780 | 1151 | putative glycosidase, PH1107-related |
| BT3798 | 1382 | putative exported fucosidase |
| BT3808 | 911 | nucleotidyltransferase family protein |
| BT3813 | 1022 | rod shape-determining protein MreB |
| BT3814 | 842 | rod shape-determining protein |
| BT3816 | 1862 | penicillin-binding protein 2 (PBP-2) |
| BT3817 | 1457 | rod shape-determining protein rodA |
| BT3820 | 1124 | putative DNA polymerase III, delta subunit |
| BT3834 | 1007 | 3-oxoacyl-[acyl-carrier-protein] synthase III |
| BT3835 | 881 | putative GTP-binding protein |
| BT3836 | 1313 | putative phosphoglycerate dehydrogenase |
| BT3837 | 767 | ABC transporter ATP-binding protein |
| BT3848 | 1382 | peptidyl-prolyl cis-trans isomerase |
| BT3849 | 1730 | hypothetical protein |
| BT3856 | 695 | conserved hypothetical protein |
| BT3864 | 1100 | tryptophanyl-tRNA synthetase |
| BT3868 | 1985 | beta-N-hexosaminidase, glycosyl hyrolase |
| | | family 20 |
| BT3872 | 1490 | ribosomal large subunit pseudouridine synthase B |
| BT3873 | 1403 | asparaginyl-tRNA synthetase |
| BT3877 | 836 | 30S ribosomal protein S2 (BS1) |
| BT3878 | 992 | elongation factor Ts (EF-Ts) |
| BT3883 | 514 | 2-hydroxyhepta-2,4-diene-1,7-dioate isomerase |
| BT3917 | 908 | putative inorganic polyphosphate/ATP-NAD |
| | | kinase |
| BT3927 | 542 | hypothetical protein |
| BT3929 | 758 | triosephosphate isomerase |
| BT3931 | 590 | GTP cyclohydrolase I |
| BT3932 | 2192 | DNA primase |
| BT3943 | 563 | conserved hypothetical protein, putative |
| | | translation factor |
| BT3945 | 968 | methionyl-tRNA formyltransferase |
| BT3966 | 791 | two-component system response regulator |
| BT3995 | 2618 | alanyl-tRNA synthetase |
| BT3996 | 968 | putative peptidase |
| BT3998 | 2243 | GTP pyrophosphokinase |
| BT4000 | 887 | conserved hypothetical protein |
| BT4001 | 890 | putative chromosome partitioning protein parB |
| BT4004 | 1136 | lipid-A-disaccharide synthase |
| BT4006 | 842 | phosphatidate cytidylyltransferase |
| BT4007 | 2090 | AAA-metalloprotease FtsH, with ATPase domain |
| BT4011 | 860 | DNA-methyltransferase |
| BT4044 | 986 | putative dolichol-P-glucose synthetase |
| BT4046 | 1556 | hypothetical protein |
| BT4099 | 1943 | 1-deoxy-D-xylulose 5-phosphate synthase |
| BT4101 | 2321 | alanine racemase |
| BT4149 | 1058 | exo-poly-alpha-D-galacturonosidase precursor |
| BT4150 | 1238 | putative rhamnogalacturonan acetylesterase |
| BT4176 | 1397 | conserved hypothetical protein, putative |
| | | cytoplasmic protein |
| BT4210 | 1112 | peptide chain release factor 1 |
| BT4234 | 569 | similar to FimX |
| BT4253 | 431 | 6,7-dimethyl-8-ribityllumazine synthase |
| BT4263 | 1010 | glyceraldehyde 3-phosphate dehydrogenase |
| BT4271 | 707 | hypothetical protein |
| BT4293 | 1745 | hypothetical protein |
| BT4302 | 665 | putative transmembrane protein |
| BT4307 | 704 | putative glycogen synthase |
| BT4308 | 848 | pantoate--beta-alanine ligase |
| BT4312 | 1274 | seryl-tRNA synthetase |
| BT4321 | 800 | 2-dehydro-3-deoxyphosphooctonate aldolase |
| BT4322 | 926 | conserved hypothetical protein, with a |
| | | diacylglycerol kinase catalytic domain |
| BT4334 | 2495 | FtsK/SpoIIIE family protein |
| BT4335 | 647 | hypothetical protein |
| BT4353 | 2639 | valyl-tRNA synthetase |
| BT4356 | 1028 | putative anti-sigma factor |
| BT4362 | 3320 | preprotein translocase SecA subunit |
| BT4366 | 668 | putative transcription regulator |
| BT4375 | 1241 | transcriptional regulator |
| BT4376 | 527 | conserved hypothetical protein |
| BT4425 | 716 | deoxyribose-phosphate aldolase |
| BT4428 | 890 | conserved hypothetical protein |
| BT4449 | 1067 | putative dehydrogenase |
| BT4483 | 773 | hypothetical protein |
| BT4490 | 572 | hypothetical protein |
| BT4504 | 674 | hypothetical protein |
| BT4522 | 527 | Type I restriction-modification enzyme |
| BT4546 | 1073 | hypothetical protein |
| BT4571 | 560 | RNA polymerase ECF-type sigma factor |
| BT4588 | 569 | peptidyl-tRNA hydrolase |
| BT4594 | 614 | putative dephospho-CoA kinase |
| BT4615 | 1916 | chaperone protein dnaK |
| BT4637 | 1019 | putative inorganic phosphate transporter |
| BT4638 | 647 | hypothetical protein |
| BT4643 | 551 | RNA polymerase ECF-type sigma factor |
| BT4685 | 515 | conserved hypothetical protein |
| BT4709 | 941 | glycosyl hydrolase |
| BT4712 | 746 | conserved hypothetical protein |
| BT4748 | 1022 | Helicase-like |
| BT4780 | 899 | conserved protein found in conjugate transposon |

### V. Control Molecules

In certain embodiments, the control molecule is not regularly present in the human diet. In certain embodiments, the control molecule is a monosaccharide or a polysaccharide, for example, a marine polysaccharide or an antibiotic or a derivative of either. In certain embodiments, the marine polysaccharide is porphyran or agarose or a derivative thereof. In certain embodiments, the antibiotic or derivative thereof is anhydrotetracycline.

In certain embodiments, the control molecule is a molecule that is not part of a common diet of a given population, or one that is found in less than about 10%, 5%, 1%, 0.1%, 0.01%, or less than about 0.001% of guts of a given population. The given population may be described geographically, for example a control molecule may be one which is not a part of a traditional North American (European, South American, African, Asian, etc.) diet. The population may also be defined in other ways, for example a subpopulation. In some cases, a control molecule is not commonly found in the diet of a first population, though it may be common in the diet of a second population. In some embodiments, a rare carbohydrate is one that is found in less than 1%, 0.1%, 0.01%, or 0.001% of guts of a population . In some cases, the control molecule is a marine carbohydrate, for example porphyran or agarose. In some cases, the control molecule is a medication, for example an antibiotic or an antibiotic derivative such as tetracycline or anhydrotetracycline. In some cases, the control molecule is a halogenated carbohydrate, such as 1-chloro-1 -deoxy-D-fructose or 1,6-dichloro-1,6-dideoxy-D-fructose. In some cases, the control molecule is one that is lacking from the North American (European, South American, African, Asian, etc.) diet. In some cases, the control molecule is one that is consumed infrequently (e.g., less than 20 times a year, 10 times a year, 9 times a year, 8 times a year, 7 times a year, 6 times a year, 5 times a year, 4 times a year, 3 times a year), on average, in the North American (European, South American, African, Asian, etc.) diet. In some cases, the control molecule is non-naturally occurring. In some cases, the control molecule is present when the temperature of the environment is within a given range.

In certain embodiments, the control molecule is a porphyran and the first and second activator are each an HTCS protein, and (i) the porphyran, when present, activates the first and second HTCS proteins, (ii) the first and second HTCS proteins, when activated, activate the first and second promoters, respectively, and (iii) the first and second promoters, when activated, direct expression of the first and second essential genes, respectively, thereby resulting in the growth and/or viability of the bacterium being dependent upon the presence of the porphyran. In certain embodiments, the bacterium is a commensal bacterium.

### VI. Modified Bacteria

A contemplated modified bacterium, for example, for use in a disclosed pharmaceutical composition or method, includes Escherichia coli, Lactococcus lactis, members of the Bacteroidetes, Firmicute. Actinobacteria. Proteobacteria or Verrucomicrobia phylum, and a bacterium of genus Bacteroides, Alistipes, Faecalibacterium, Parabacteroides, Prevotella, Roseburia, Ruminococcus, Clostridium, Oscillibacter, Gemmiger, Barnesiella, Dialister, Parasutterella, Phascolarctobacterium, Propionibacterium, Sutterella, Blautia, Paraprevotella, Coprococcus, Odoribacter, Spiroplasma, Anaerostipes, or Akkermansia. A contemplated bacterium, for example, for use in a disclosed pharmaceutical composition or method, may be of the Bacteroides genus, i.e., may be a Bacteroides species bacterium.

Exemplary Bacteroides species include B. acidifaciens, B. amylophilus, B. asaccharolyticus, B. barnesiaes, B. bivius, B. buccae, B. buccalis, B. caccae, B. caecicola, B. caecigallinarum, B. capillosus, B. capillus, B. cellulosilyticus, B. cellulosolvens, B. chinchilla, B. clarus, B. coagulans, B. coprocola, B. coprophilus, B. coprosuis, B. corporis, B. denticola, B. disiens, B. distasonis, B. dorei, B. eggerthii, B. endodontalis, B. faecichinchillae, B. faecis, B. finegoldii, B. fluxus, B. forsythus, B. fragilis, B. furcosus, B. galacturonicus, B. gallinaceum, B. gallinarum, B. gingivalis, B. goldsteinii, B. gracilis, B. graminisolvens, B. helcogenes, B. heparinolyticus, B. hypermegas, B. intermedius, B. intestinalis, B. johnsonii, B. levvi, B. loescheii, B. luti, B. macacae, B. massiliensis, B. melaninogenicus, B. merdae, B. microfusus, B. multiacidus, B. nodosus, B. nordii, B. ochraceus, B. oleiciplenus, B. oralis, B. oris, B. oulorum, B. ovatus, B. paurosaccharolyticus, B. pectinophilus, B. pentosaceus, B. plebeius, B. pneumosintes, B. polypragmatus, B. praeacutus, B. propionicifaciens, B. putredinis, B. pyogenes, B. reticulotermitis, B. rodentium, B. ruminicola, B. salanitronis, B. salivosus, B. salyersiae, B. sartorii, B. sediment, B. splanchnicus, B. stercorirosoris, B. stercoris, B. succinogenes, B. suis, B. tectus, B. termitidis, B. thetaiotaomicron, B. uniformis, B. ureolyticus, B. veroralis, B. vulgatus, B. xylanisolvens, B. xylanolyticus, or B. zoogleofonnans.

As used herein, the term “species” refers to a taxonomic entity as conventionally defined by genomic sequence and phenotypic characteristics. A “strain” is a particular instance of a species that has been isolated and purified according to conventional microbiological techniques. The present disclosure encompasses derivatives of the disclosed bacterial strains. The term “derivative” includes daughter strains (progeny) or stains cultured (sub-cloned) from the original but modified in some way (including at the genetic level), without altering negatively a biological activity of the strain.

In certain embodiments, a contemplated modified bacterium is of a genus that makes up more than 0.1%, 0.5%, 1%, 5%, 10%, 20%, 30%, or 40% of the total culturable microbes in the feces of a subject to be treated, or in the feces of an average human. In certain embodiments, a contemplated modified bacterium is of a genus that is detected at a level greater than 10¹², 10¹¹, 10¹⁰, 10⁹, 10⁸, 10⁷ colony forming units per gram of feces of a subject to be treated, or per gram of feces of an average human. In certain embodiments, a contemplated modified bacterium is of a genus that makes up more than 0.1%, 0.5%, 1%, 5%, 10%, 20%, 30%, or 40% of the gut microbiome of a subject to be treated, or of the gut microbiome of an average human. Human gut or feces microbiome composition may be assayed by any technique known in the art, including 16S ribosomal sequencing. Bacteroidesis the most naturally abundant genus in the human gut (Huttenhower et al. (2012) NATURE 486.7402:207).

rRNA, 16S rDNA, 16S rRNA, 16S, 18S, 18S rRNA, and 18S rDNA refer to nucleic acids that are components of, or encode for, components of the ribosome. There are two subunits in the ribosome termed the small subunit (SSU) and large subunit (LSU). rDNA genes and their complementary RNA sequences are widely used for determination of the evolutionary relationships amount organisms as they are variable, vet sufficiently conserved to allow cross- organism molecular comparisons.

16S rDNA sequence (approximately 1542 nucleotides in length) of the 30S SSU can be used, in embodiments, for molecular-based taxonomic assignments of prokaryotes and the 18S rDNA sequence (approximately 1869 nucleotides in length) of 40S SSU may be used for eukaryotes. For example, 16S sequences may be used for phylogenetic reconstruction as they are general highly conserved but contain specific hypervariable regions that harbor sufficient nucleotide diversity to differentiate genera and species of most bacteria. Although 16S rDNA sequence data has been used to provide taxonomic classification, closely related bacterial strains that are classified within the same genus and species, may exhibit distinct biological phenotypes.

The identity of contemplated bacterial species or strains may be characterized by 16S rRNA or full genome sequence analysis. For example, in certain embodiments, contemplated bacterial strains may comprise a 16S rRNA or genomic sequence having a certain % identity to a reference sequence.

Sequence identity may be determined in various ways that are within the skill in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EVOL. 36, 290-300; Altschul et al., (1997) NUCLEIC ACIDS RES. 25:3389-3402, incorporated by reference) are tailored for sequence similarity searching. For a discussion of basic issues in searching sequence databases see Altschul et al., (1994) NATURE GENETICS 6:119-129, which is fully incorporated by reference. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919, fully incorporated by reference). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent Blastp parameter settings may be Q=9; R=2; wink=1; and gapw=32. Searches may also be conducted using the NCBI (National Center for Biotechnology Information) BLAST Advanced Option parameter (e.g.: -G, Cost to open gap [Integer]: default=5 for nucleotides/11 for proteins; -E, Cost to extend gap [Integer]: default=2 for nucleotides/1 for proteins; -q, Penalty for nucleotide mismatch [Integer]: default=−3; -r, reward for nucleotide match [Integer]: default=1; -e, expect value [Real]: default=10; -W, wordsize [Integer]: default=11 for nucleotides/28 for megablast/3 for proteins; -y, Dropoff (X) for blast extensions in bits: default=20 for blastn/7 for others; -X, X dropoff value for gapped alignment (in bits): default=15 for all programs, not applicable to blastn; and -Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

In certain embodiments, a contemplated modified bacterium is capable of stably colonizing the human gut. A disclosed bacterium may, e.g., upon administration to a human subject, result in an abundance greater than 10¹², 10¹¹, 10¹⁰, 10⁹, 10⁸, or 10⁷ cfu per gram of fecal content. For example, administration of about 10³, about 10⁴, about 10⁵, about 10⁶, about 10⁷, about 10⁸, about 10⁹, about 10¹⁰, about 10¹¹, or about 10¹² cells of a disclosed bacterium to a human subject may result in an abundance greater than 10¹², 10¹¹, 10¹⁰, 10⁹, 10⁸, or 10⁷ cfu per gram of fecal content with 12 hours, 24 hours, 36 hours, 48 hours, 60 hours, or 72 hours of administration.

A disclosed bacterium may, e.g., have been modified to colonize the human gut with increased abundance, stability, predictability or ease of initial colonization relative to a similar or otherwise identical bacterium that has not been modified. For example, a contemplated bacterium may be modified to increase its ability to utilize a privileged nutrient as carbon source. A “privileged nutrient” is defined as a molecule or set of molecules that can be consumed to aid in the proliferation of a particular bacterial strain while providing proliferation assistance to no more than 1% of the other bacteria in the gut. Accordingly, in certain embodiments, a modified bacterium has the ability to consume the privileged nutrient to sustain its colonization and expand in the gut of a subject to a predictably high abundance, even in the absence of other carbon or energy sources, while most other bacteria in the gut of the subject do not. Exemplary privileged nutrients include, e.g., a marine polysaccharide, e.g., a porphyran. As the skilled artisan will recognize, contemplated privileged nutrients may overlap with contemplated control molecules for a given bacterium and subject.

For example, in certain embodiments, a bacterium may comprise all or a portion of a polysaccharide utilization locus (PUL), a mobile genetic element that confers the ability to consume a carbohydrate, e.g., a privileged nutrient, upon a bacterium. An exemplary porphyran consumption PUL is the PUL from the porphyran-consuming Bacteroides strain NB001 depicted in SEQ ID NO: 14. Accordingly, in certain embodiments, a modified bacterium comprises SEQ ID NO: 14, or a functional fragment or variant thereof. In certain embodiments, a modified bacterium comprises a nucleotide sequence having at least 80%, 85%, 86%, 87%, 88%, 89%, 90%, 91%, 92%, 93%, 94%, 95%, 96%, 97%, 98%, or 99% identity to SEQ ID NO: 14, or a functional fragment or variant thereof.

Other exemplary PULs are those from the agarose-consuming Bacteroides strain NB002 provided in SEQ ID NO: 15 and NB003 provided in SEQ ID NO: 16. Accordingly, in certain embodiments, a modified bacterium comprises SEQ ID NO: 15 or 16, or a functional fragment or variant thereof. In certain embodiments, a modified bacterium comprises a nucleotide sequence having at least 80%, 85%, 86%, 87%, 88%, 89%, 90%, 91%, 92%, 93%, 94%, 95%, 96%, 97%, 98%, or 99% identity to SEQ ID NO: 15 or 16, or a functional fragment or variant thereof.

Additional exemplary bacterial modifications to increase abundance in the gut of a subject, privileged nutrients, transgenes that increase the ability of a bacterium to utilize a privileged nutrient. PULs, and other methods and compositions for modulating the growth of a modified bacterium are described in International (PCT) Patent Publication No. WO2018112194.

In certain embodiments, a disclosed transgene or nucleic acid comprising an heterologous nucleotide sequence is operably linked to at least one promoter, e.g., a phage-derived promoter. The term “operably linked” refers to a linkage of polynucleotide elements in a functional relationship. A nucleic acid sequence is “operably linked” when it is placed into a functional relationship with another nucleic acid sequence. For instance, a promoter or enhancer is operably linked to a gene if it affects the transcription of the gene. Operably linked nucleotide sequences are typically contiguous. However, as enhancers generally function when separated from the promoter by several kilobases and intronic sequences may be of variable lengths, some polynucleotide elements may be operably linked but not directly flanked and may even function in trans from a different allele or chromosome. In certain embodiments, the promoter comprises the consensus sequence GTTAA(n)₄₋₇GTTAA(n)₃₄₋₃₈TA(n)₂TTTG. In certain embodiments, the promoter comprises SEQ ID NO: 48, SEQ ID NO: 49, or SEQ ID NO: 50, or a functional fragment thereof, or a nucleotide sequence having at least 80%, 85%, 86%, 87%, 88%, 89%, 90%, 91%, 92%, 93%, 94%, 95%, 96%, 97%, 98%, or 99% identity to SEQ ID NO: 48, SEQ ID NO: 49, or SEQ ID NO: 50, or a functional fragment thereof. Additional exemplary phage-derived promoters are described in International (PCT) Patent Publication No. WO2017184565.

In certain embodiments, the bacterium further comprising one or more transgenes encoding a protein homologous to a starch binding protein such as SusC or SusD, e.g., SEQ ID NO: 20 or 21, or a functional fragment or variant thereof. In certain embodiments, the transgene encodes one or more of SEQ ID NO: 20 and 21, or a functional fragment thereof, or a protein having at least 80%, 85%, 86%, 87%, 88%, 89%, 90%, 91%, 92%, 93%, 94%, 95%, 96%, 97%, 98%, or 99% identity to SEQ ID NO: 20 or 21, or a functional fragment thereof.

In certain embodiments, the bacterium further comprises a therapeutic transgene. In some cases, the therapeutic transgene may be gad65, il10, il22, TNF-α, nags, add, xapA, deoD, xdhA, xdhB, xdhC, mtr, a propionate transporter, a kynurenine transporter, a bile salt transporter, an ammonia transporter, a GABA transporter, PheP or AroP. In some cases, the bacterium comprises a diagnostic transgene. In some cases, the diagnostic transgene is TtrR/TtrS. In some cases, the bacterium further comprises an outer membrane import protein.

In certain embodiments, a disclosed transgene or nucleic acid is on a plasmid, on a bacterial artificial chromosome, and/or are genomically integrated. When a bacterium comprises one or more transgenes or nucleic acids encoding multiple proteins, it is contemplated that the open reading frames encoding two or more of the proteins may, e.g., be present in a single operon.

In certain embodiments, a disclosed gene (e.g., essential gene or transgene) or nucleic acid is operably linked to at least one ribosome binding site (RBS). Exemplary RBSs include those comprising the nucleotide sequence of any one of SEQ ID NOs: 47, 74, 75, 76, 77, 84, or 85, or a nucleotide sequence having at least 80%, 85%, 86%, 87%, 88%, 89%, 90%, 91%, 92%, 93%, 94%, 95%, 96%, 97%, 98%, or 99% identity to any one of SEQ ID NOs: 47, 74, 75, 76, 77, 84, or 85, or a functional fragment or variant of any of the foregoing nucleotide sequences.

It is contemplated that a bacterium may comprise a nucleic acid comprising the nucleotide sequence of any one of SEQ ID NOs: 47, 74, 75, 76, 77, 84, or 85, or a functional fragment or variant thereof, or a nucleotide sequence having at least 80%, at least 85%, at least 90%, at least 91%, at least 92%, at least 93%, at least 94%, at least 95%, at least 96%, at least 97%, at least 98%, or at least 99% identity to any one of SEQ ID NOs: 47, 74, 75, 76, 77, 84, or 85, or a functional fragment or variant thereof.

It is contemplated that a bacterium may comprise a protein comprising the amino acid sequence of any one of SEQ ID NOs: 39, 43, 53, 54, 59, or 64-71, or a functional fragment or variant thereof, or an amino acid sequence having at least 80%, at least 85%, at least 90%, at least 91%, at least 92%, at least 93%, at least 94%, at least 95%, at least 96%, at least 97%, at least 98%, or at least 99% identity to any one of SEQ ID NOs: 39, 43, 53, 54, 59, or 64-71, or a functional fragment or variant thereof.

It is contemplated that a bacterium may comprise one or more nucleic acids comprising a nucleotide sequence encoding the amino acid sequence of any one of SEQ ID NOs: 39, 43, 53, 54, 59, or 64-71, or a functional fragment or variant thereof, or an amino acid sequence having at least 80%, at least 85%, at least 90%, at least 91%, at least 92%, at least 93%, at least 94%, at least 95%, at least 96%, at least 97%, at least 98%, or at least 99% identity to any one of SEQ ID NOs: 39, 43, 53, 54, 59, or 64-71, or a functional fragment or variant thereof.

It is contemplated that a bacterium may comprise one or more nucleic acids comprising the nucleotide sequence of any one of SEQ ID NOs: 29, 30, 31, 34, 35, 36, 37, 40, 55, 56, 60, 61, or 72, or a functional fragment or variant thereof, or a nucleotide sequence having at least 80%, at least 85%, at least 90%, at least 91%, at least 92%, at least 93%, at least 94%, at least 95%, at least 96%, at least 97%, at least 98%, or at least 99% identity to any one of SEQ ID NOs: 29, 30, 31, 34, 35, 36, 37, 40, 55, 56, 60, 61, or 72, or a functional fragment or variant thereof.

It is contemplated that a bacterium may comprise (i) an HTCS that is activated by porphyran comprising the amino acid of SEQ ID NO: 19, or a functional fragment or variant thereof, or an amino acid sequence having at least 80%, at least 85%, at least 90%, at least 91%, at least 92%, at least 93%, at least 94%, at least 95%, at least 96%, at least 97%, at least 98%, or at least 99% identity to SEQ ID NO: 19, or a functional fragment or variant thereof; (ii) a promoter that is activated by the HTCS comprising the nucleotide sequence of SEQ ID NO: 73, or a functional fragment or variant thereof, or a nucleotide sequence having at least 80%, at least 85%, at least 90%, at least 91%, at least 92%, at least 93%, at least 94%, at least 95%, at least 96%, at least 97%, at least 98%, or at least 99% identity to SEQ ID NO: 73, or a functional fragment or variant thereof; and (iii) an essential gene (e.g., an argS gene) that is operably linked to the promoter. In certain embodiments, the essential gene (e.g., the argS gene) is operably linked to a ribosome binding site (RBS) comprising the nucleotide sequence of SEQ ID NO: 47, or a functional fragment or variant thereof, or a nucleotide sequence having at least 80%, at least 85%, at least 90%, at least 91%, at least 92%, at least 93%, at least 94%, at least 95%, at least 96%, at least 97%, at least 98%, or at least 99% identity to SEQ ID NO: 47, or a functional fragment or variant thereof.

It is contemplated that a bacterium may comprise (i) an HTCS that is activated by porphyran comprising the amino acid of SEQ ID NO: 59, or a functional fragment or variant thereof, or an amino acid sequence having at least 80%, at least 85%, at least 90%, at least 91%, at least 92%, at least 93%, at least 94%, at least 95%, at least 96%, at least 97%, at least 98%, or at least 99% identity to SEQ ID NO: 59, or a functional fragment or variant thereof; (ii) a promoter that is activated by the HTCS comprising the nucleotide sequence of SEQ ID NO: 45, or a functional fragment or variant thereof, or a nucleotide sequence having at least 80%, at least 85%, at least 90%, at least 91%, at least 92%, at least 93%, at least 94%, at least 95%, at least 96%, at least 97%, at least 98%, or at least 99% identity to SEQ ID NO: 45, or a functional fragment or variant thereof; and (iii) an essential gene (e.g., a lytB gene) that is operably linked to the promoter. In certain embodiments, the essential gene (e.g., the lytB gene) is operably linked to a ribosome binding site (RBS) comprising the nucleotide sequence of SEQ ID NO: 84, or a functional fragment or variant thereof, or a nucleotide sequence having at least 80%, at least 85%, at least 90%, at least 91%, at least 92%, at least 93%, at least 94%, at least 95%, at least 96%, at least 97%, at least 98%, or at least 99% identity to SEQ ID NO: 84, or a functional fragment or variant thereof.

It is contemplated that a bacterium may comprise (i) a first HTCS that is activated by porphyran comprising the amino acid of SEQ ID NO: 19, or a functional fragment or variant thereof, or an amino acid sequence having at least 80%, at least 85%, at least 90%, at least 91%, at least 92%, at least 93%, at least 94%, at least 95%, at least 96%, at least 97%, at least 98%, or at least 99% identity to SEQ ID NO: 19, or a functional fragment or variant thereof; (ii) a first promoter that is activated by the first HTCS comprising the nucleotide sequence of SEQ ID NO: 73, or a functional fragment or variant thereof, or a nucleotide sequence having at least 80%, at least 85%, at least 90%, at least 91%, at least 92%, at least 93%, at least 94%, at least 95%, at least 96%, at least 97%, at least 98%, or at least 99% identity to SEQ ID NO: 73, or a functional fragment or variant thereof; (iii) a first essential gene (e.g., an argS gene) that is operably linked to the first promoter; (iv) a second HTCS that is activated by porphyran comprising the amino acid of SEQ ID NO: 59, or a functional fragment or variant thereof, or an amino acid sequence having at least 80%, at least 85%, at least 90%, at least 91%, at least 92%, at least 93%, at least 94%, at least 95%, at least 96%, at least 97%, at least 98%, or at least 99% identity to SEQ ID NO: 59, or a functional fragment or variant thereof; (v) a second promoter that is activated by the second HTCS comprising the nucleotide sequence of SEQ ID NO: 45, or a functional fragment or variant thereof, or a nucleotide sequence having at least 80%, at least 85%, at least 90%, at least 91%, at least 92%, at least 93%, at least 94%, at least 95%, at least 96%, at least 97%, at least 98%, or at least 99% identity to SEQ ID NO: 45, or a functional fragment or variant thereof; and (vi) a second essential gene (e.g., a lytB gene) that is operably linked to the second promoter. In certain embodiments, the first essential gene (e.g., the argS gene) is operably linked to a first ribosome binding site (RBS) comprising the nucleotide sequence of SEQ ID NO: 47, or a functional fragment or variant thereof, or a nucleotide sequence having at least 80%, at least 85%, at least 90%, at least 91%, at least 92%, at least 93%, at least 94%, at least 95%, at least 96%, at least 97%, at least 98%, or at least 99% identity to SEQ ID NO: 47, or a functional fragment or variant thereof. In certain embodiments, the second essential gene (e.g., the lytB gene) is operably linked to a second ribosome binding site (RBS) comprising the nucleotide sequence of SEQ ID NO: 84, or a functional fragment or variant thereof, or a nucleotide sequence having at least 80%, at least 85%, at least 90%, at least 91%, at least 92%, at least 93%, at least 94%, at least 95%, at least 96%, at least 97%, at least 98%, or at least 99% identity to SEQ ID NO: 84, or a functional fragment or variant thereof.

### VII. Methods

In another aspect, the disclosure relates to a method for reducing the growth and/or viability of a bacterium (e.g., a commensal bacterium) in the absence of a control molecule. The method includes genetically modifying the bacterium to comprise a first activator that is activated by the control molecule, a first promoter that is activated by the first activator, and a first essential gene that is operably linked to the first promoter. In certain embodiments, the method further includes genetically modifying the bacterium to comprise a second activator that is activated by the control molecule, a second promoter that is activated by the second activator, and a second essential gene that is operably linked to the second promoter. In certain embodiments, the first promoter is not activated by the second activator and the second promoter is not activated by the first activator. Incorporating different activator/promoter pairs that do not cross-activate provides redundancy and reduces the escape rate.

Accordingly, to further reduce the growth and/or viability of a bacterium in the absence of a control molecule, a third activator that is activated by the control molecule may be introduced. Thus, the method can further include genetically modifying the bacterium to comprise a third activator that is activated by the control molecule, a third promoter that is activated by the third activator, and a third essential gene that is operably linked to the third promoter. In certain embodiments, the third promoter is not activated by the first or second activator and the third promoter is not activated by the first or second activator. Incorporating additional activator/promoter pairs provides additional redundancy and further reduces the escape rate.

In certain embodiments, the method further includes genetically modifying the bacterium to comprise one or more transgenes encoding the first, second, and/or third activator.

The disclosure also relates to a method of colonizing the gut of a subject, the method comprising administering the bacterium or the pharmaceutical composition as described herein. Strategies for increasing colonization of the gut are discussed in more detail below.

### VIII. Pharmaceutical Compositions/Units

A bacterium disclosed herein may be combined with pharmaceutically acceptable excipients to form a pharmaceutical composition, which can be administered to a patient by any means known in the art. As used herein, the term “pharmaceutically acceptable excipient” is understood to mean one or more of a buffer, carrier, or excipient suitable for administration to a subject, for example, a human subject, without excessive toxicity, irritation, allergic response, or other problem or complication, commensurate with a reasonable benefit/risk ratio. The excipient(s) should be “acceptable” in the sense of being compatible with the other ingredients of the formulations and not deleterious to the recipient.

Pharmaceutically acceptable excipients include buffers, solvents, dispersion media, coatings, isotonic and absorption delaying agents, and the like, that are compatible with pharmaceutical administration. Pharmaceutically acceptable excipients also include fillers, binders, disintegrants, glidants, lubricants, and any combination(s) thereof. For further examples of excipients, carriers, stabilizers and adjuvants, see, e.g., Handbook of Pharmaceutical Excipients, 8^{th} Ed., Edited by P. J. Sheskey, W. G. Cook, and C. G. Cable, Pharmaceutical Press, London, UK [2017]. The use of such media and agents for pharmaceutically active substances is known in the art.

Contemplated bacteria may be used in disclosed compositions in any form, e.g., a stable form, as known to those skilled in the art, including in a lyophilized state (with optionally one or more appropriate cryoprotectants), frozen (e.g., in a standard or super-cooled freezer), spray dried, and/or freeze dried. A “stable” formulation or composition is one in which the biologically active material therein essentially retains its physical stability, chemical stability, and/or biological activity upon storage. Stability can be measured at a selected temperature and humidity conditions for a selected time period. Trend analysis can be used to estimate an expected shelf life before a material has actually been in storage for that time period. For live bacteria, for example, stability may be defined as the time it takes to lose 1 log of cfu/g dry formulation under predefined conditions of temperature, humidity and time period.

A bacterium disclosed herein may be combined with one or more cryoprotectants. Exemplary cryoprotectants include fructoligosaccharides (e.g., raftilose®), trehalose, maltodextrin, sodium alginate, proline, glutamic acid, glycine (e.g., glycine betaine), mono-, di-, or polysaccharides (such as glucose, sucrose, maltose, lactose), polyols (such as mannitol, sorbitol, or glycerol), dextran, DMSO, methylcellulose, propylene glycol, polyvinylpyrrolidone, non-ionic surfactants such as Tween 80, and/or any combinations thereof.

A pharmaceutical composition should be formulated to be compatible with its intended route of administration. Contemplated bacterial compositions disclosed herein can be prepared by any suitable method and can be formulated into a variety of forms and administered by a number of different means. Contemplated compositions can be administered orally, rectally, or enterally, in formulations containing conventionally acceptable carriers, adjuvants, and vehicles as desired. As used herein, “rectal administration” is understood to include administration by enema, suppository. or colonoscopy. A disclosed pharmaceutical composition may, e.g., be suitable for bolus administration or bolus release. In an exemplary embodiment, a disclosed bacterial composition is administered orally.

Solid dosage forms for oral administration include capsules, tablets, caplets, pills, troches, lozenges, powders, and granules. A capsule typically comprises a core material comprising a bacterial composition and a shell wall that encapsulates the core material. In some embodiments the core material comprises at least one of a solid, a liquid, and an emulsion. In some embodiments the shell wall material comprises at least one of a soft gelatin, a hard gelatin, and a polymer. Suitable polymers include, but are not limited to: cellulosic polymers such as hydroxypropyl cellulose, hydroxyethyl cellulose, hydroxypropyl methyl cellulose (HPMC), methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, such as those formed from acrylic acid, methacry lic acid, methyl acrylate, ammonio methylacrylate, ethyl acrylate, methyl methacrylate and/or ethyl methacry late (e.g., those copolymers sold under the trade name “Eudragit®”); vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac (purified lac). In some embodiments at least one polymer functions as a taste-masking agent.

Tablets, pills, and the like can be compressed, multiply compressed, multiply layered, and/or coated. A contemplated coating can be single or multiple. In one embodiment, a contemplated coating material comprises at least one of a saccharide, a polysaccharide, and glycoproteins extracted from at least one of a plant, a fungus, and a microbe. Non-limiting examples include corn starch, wheat starch, potato starch, tapioca starch, cellulose, hemicellulose, dextrans, maltodextrin, cyclodextrins, inulins, pectin, mannans, gum arabic, locust bean gum, mesquite gum, guar gum, gum karaya, gum ghatti, tragacanth gum, funori, carrageenans, porphyrans, agar, alginates, chitosans, or gellan gum. In some embodiments a contemplated coating material comprises a protein. In some embodiments a contemplated coating material comprises at least one of a fat and an oil. In some embodiments the at least one of a fat and an oil is high temperature melting. In some embodiments the at least one of a fat and an oil is hydrogenated or partially hydrogenated. In some embodiments the at least one of a fat and an oil is derived from a plant. In some embodiments the at least one of a fat and an oil comprises at least one of glycerides, free fatty acids, and fatty acid esters. In some embodiments a contemplated coating material comprises at least one edible wax. A contemplated edible wax can be derived from animals, insects, or plants. Non-limiting examples include beeswax, lanolin, bayberry wax, carnauba wax, and rice bran wax. Tablets and pills can additionally be prepared with enteric or reverse-enteric coatings.

Alternatively, powders or granules embodying a bacterial composition disclosed herein can be incorporated into a food product. In some embodiments a contemplated food product is a drink for oral administration. Non-limiting examples of a suitable drink include water, fruit juice, a fruit drink, an artificially flavored drink, an artificially sweetened drink, a carbonated beverage, a sports drink, a liquid diary product, a shake, an alcoholic beverage, a caffeinated beverage, infant formula and so forth. Other suitable means for oral administration include aqueous and nonaqueous solutions, emulsions, suspensions and solutions and/or suspensions reconstituted from non-effervescent granules, containing at least one of suitable solvents, preservatives, emulsifying agents, suspending agents, diluents, sweeteners, coloring agents, and flavoring agents.

Pharmaceutical compositions containing a bacterium disclosed herein can be presented in a unit dosage form, i.e .. a pharmaceutical unit. A composition, e.g., a pharmaceutical unit provided herein, may include any appropriate amount of bacterium, measured either by total mass or by colony forming units of the bacteria.

For example, a disclosed pharmaceutical composition or unit may include from about 10³ cfus to about 10¹² cfus, about 10⁶ cfus to about 10¹² cfus, about 10⁷ cfus to about 10¹² cfus, about 10⁸ cfus to about 10¹² cfus, about 10⁹ cfus to about 10¹² cfus, about 10¹⁰ cfus to about 10¹² cfus, about 10¹¹ cfus to about 10¹² cfus, about 10³ cfus to about 10¹¹ cfus, about 10⁶ cfus to about 10¹¹ cfus, about 10⁷ cfus to about 10¹¹ cfus, about 10⁸ cfus to about 10¹¹ cfus, about 10⁹ cfus to about 10¹¹ cfus, about 10¹⁰ cfus to about 10¹¹ cfus, about 10³ cfus to about 10¹⁰ cfus, about 10⁶ cfus to about 10¹⁰ cfus, about 10⁷ cfus to about 10¹⁰ cfus, about 10⁸ cfus to about 10¹⁰ cfus, about 10⁹ cfus to about 10¹⁰ cfus, about 10³ cfus to about 10⁹ cfus, about 10⁶ cfus to about 10⁹ cfus, about 10⁷ cfus to about 10⁹ cfus, about 10⁸ cfus to about 10⁹ cfus, about 10³ cfus to about 10⁸ cfus, about 10⁶ cfus to about 10⁸ cfus, about 10⁷ cfus to about 10⁸ cfus, about 10³ cfus to about 10⁷ cfus, about 10⁶ cfus to about 10⁷ cfus, or about 10³ cfus to about 10⁶ cfus of each bacterial strain, or may include about 10³ cfus, about 10⁶ cfus, about 10⁷ cfus, about 10⁸ cfus, about 10⁹ cfus, about 10¹⁰ cfus, about 10¹¹ cfus, or about 10¹² cfus of bacteria.

In certain embodiments, the pharmaceutical compositions or unit may further comprise a control molecule. In certain embodiments, the pharmaceutical compositions comprises the control molecule in an amount sufficient to preserve viability of the bacterium when administered to a subject. For example, the control molecule may be present in an amount from about 10 mg to about 100 g per dose. In certain embodiments, the control molecule may be present in an amount from about 10 mg to about 10 g per dose, from about 10 mg to about 1 g per dose, from about 10 mg to about 100 mg per dose, from about 100 mg to about 1 g per dose, from about 100 mg to about 10 g per dose, from about 100 mg to about 100 g per dose, from about 100 mg to about 100 g per dose, from about 1 g to about 10 g per dose, from about 1 g to about 100 g per dose, or from about 10 g to about 100 g per dose.

### IX. Therapeutic Uses

In some embodiments, this disclosure provides a method of treating a subject with a disease or disorder, comprising: administering to the subject a bacterium engineered to require a control molecule for viability. The bacterium may express a therapeutic transgene. The bacterium may be maintained in the subject by administration of a control molecule to the subject for a sufficient time to treat the disease or disorder.

In some embodiments a method of diagnosing or monitoring a subject with a disease or disorder, may comprise: administering to the subject a bacterium engineered to require a control molecule for viability. The bacterium may express a diagnostic transgene and be maintained in the subject by administration of a control molecule to the subject for a sufficient time to diagnose or monitor the disease or disorder. In some cases, the bacterium may be incapable of person to person transmission, or organism to organism transmission. The control molecule and the bacterium may be administered to the subject orally. In some cases, the subject is a human. In some examples, the control molecule bacterium cannot be detected in the subject at least one day, two days, three days, four days, one week, or two weeks after a last administration.

As used herein, “treat”, “treating” and “treatment” mean the treatment of a disease in a subject, e.g., in a human. This includes: (a) inhibiting the disease, i.e., arresting its development; and (b) relieving the disease, i.e., causing regression of the disease state. As used herein, the terms “subject” and “patient” refer to a bacterium to be treated by the methods and compositions described herein. Such organisms preferably include, but are not limited to, mammals. e.g., human, a companion animal (e.g., dog, cat, or rabbit), or a livestock animal (for example, cow, sheep, pig, goat, horse, donkey, and mule, buffalo, oxen, or camel).

It will be appreciated that the exact dosage of a pharmaceutical composition, or bacterium is chosen by an individual physician in view of the patient to be treated, in general, dosage and administration are adjusted to provide an effective amount of the bacterial agent to the patient being treated. As used herein, the “effective amount” refers to the amount necessary to elicit a beneficial or desired biological response. An effective amount can be administered in one or more administrations, applications or dosages and is not intended to be limited to a particular formulation or administration route. As will be appreciated by those of ordinary skill in this art, the effective amount of a pharmaceutical unit, pharmaceutical composition, or bacterial strain may vary depending on such factors as the desired biological endpoint, the drug to be delivered, the target tissue, the route of administration, etc. Additional factors which may be taken into account include the severity of the disease state; age, weight and gender of the patient being treated; diet, time and frequency of administration; drug combinations; reaction sensitivities; and tolerance/response to therapy.

Contemplated methods may further comprise administrating a control molecule and/or a privileged nutrient to the subject to support colonization of the bacterium. Exemplary privileged nutrients include marine polysaccharides, e.g., a porphyran. For example, a disclosed privileged nutrient may be administered to the subject prior to, at the same time as, or after a disclosed bacterium.

Contemplated methods may comprise administration of a disclosed bacterium or pharmaceutical composition to a subject every 12 hours, 24 hours, day, 2 days, 3 days, 4 days, 5 days, 6 days, 7 days, week, 2 weeks, 3 weeks, 4 weeks, month, 2 months, 3 months, 4 months, 5 months, or 6 months. In certain embodiments, the time between consecutive administrations of a disclosed bacterium or pharmaceutical composition to a subject is greater than 12 hours, 24 hours, 36 hours, 48 hours, 2 days, 3 days, 4 days, 5 days, 6 days, 7 days, 1 week, 2 weeks, 3 weeks, or 4 weeks.

In certain embodiments, a disclosed bacterium and a disclosed control molecule and/or privileged nutrient, e.g., a marine polysaccharide, e.g., a porphyran are administered to a subject with the same frequency. For example, the bacterium and the privileged nutrient may both be administered to the subject every 8 hours, 12 hours, 24 hours, day, 2 days, 3 days, 4 days, 5 days, 6 days, 7 days, week, 2 weeks, 3 weeks, 4 weeks, month, 2 months, 3 months, 4 months, 5 months, or 6 months. In certain embodiments, a disclosed bacterium and a disclosed control molecule and/or privileged nutrient, e.g., a marine polysaccharide, e.g., a porphyran, are administered to a subject with a different frequency. For example, the bacterium may be administered to the subject every 8 hours, 12 hours, 24 hours, day, 2 days, 3 days, 4 days, 5 days, 6 days, 7 days, week, 2 weeks, 3 weeks, 4 weeks, month, 2 months, 3 months, 4 months, 5 months, or 6 months, and the control molecule and/or privileged nutrient may be administered to the subject every 8 hours, 12 hours, 24 hours, day, 2 days, 3 days, 4 days, 5 days, 6 days, 7 days, week, 2 weeks, 3 weeks, 4 weeks, month, 2 months, 3 months, 4 months, 5 months, or 6 months. For example, in certain embodiments, the bacterium may be administered to the subject every week, 2 weeks, 3 weeks, 4 weeks, month, 2 months, 3 months, 4 months, 5 months, or 6 months, and the privileged nutrient may be administered to the subject every 8 hours, 12 hours, 24 hours, day, 2 days, 3 days, 4 days, 5 days, 6 days, or 7 days.

Methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. In certain embodiments, a side effect of a first and/or second treatment is reduced because of combined administration.

In certain embodiments, the disclosure relates to a method of clearing a therapeutic bacterium from a subject, wherein the bacterium encodes a therapeutic transgene that has reduced function (e.g., the therapeutic transgene becomes mutated thereby reducing or eliminating its therapeutic function). In certain embodiment, the reduction in function is a complete reduction, such that the therapeutic transgene is non-functional.

A bacterium having a therapeutic transgene with reduced function may have a reproductive advantage and outcompete bacteria carrying a functional therapeutic transgene. Accordingly, it is contemplated that in certain embodiments, a subject may be administered a control molecule (and optionally a bacterium as disclosed herein) for a first period of time (e.g., 6 months, 5 months, 4 months, 3 months, 2 months, 1 month, 2 weeks, 1 week), followed by a second period of time (e.g., 1 week, 2 weeks, 3 weeks, 1 month, 2 months) in which the subject does not receive the control molecule. During the second period of time, the bacterium comprising the reduced-function therapeutic transgene will be cleared from the subject. In certain embodiments, the method further includes a third period of time, after the bacterium comprising the reduced-function therapeutic transgene is cleared from the subject, in which the subject is administered a bacterium comprising a functional therapeutic transgene according to any of the treatment regimens described herein.

### Kits

In some embodiments a kit is provided comprising a bacterium as described herein. In one aspect such a kit comprises a bacterium as described herein; and a control molecule that is required for expression of one or more essential genes in the bacterium.

Throughout the description, where compositions are described as having, including, or comprising specific components, or where processes and methods are described as having, including, or comprising specific steps, it is contemplated that, additionally, there are compositions of the present disclosure that consist essentially of, or consist of, the recited components, and that there are processes and methods according to the present disclosure that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present disclosure, whether explicit or implicit herein. For example, where reference is made to a particular compound, that compound can be used in various embodiments of compositions of the present disclosure and/or in methods of the present disclosure, unless otherwise understood from the context. In other words, within this application, embodiments have been described and depicted in a way that enables a clear and concise application to be written and drawn, but it is intended and will be appreciated that embodiments may be variously combined or separated without parting from the present teachings and disclosure. For example, it will be appreciated that all features described and depicted herein can be applicable to all aspects of the disclosure described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present disclosure also includes the specific quantitative value itself, unless specifically stated otherwise. As used herein, the term “about” refers to a +10% variation from the nominal value, or to a +10× variation on a log scale, unless otherwise indicated or inferred.

It should be understood that the order of steps or order for performing certain actions is immaterial so long as the present disclosure remains operable. Moreover, two or more steps or actions may be conducted simultaneously.

The use of any and all examples, or exemplary language herein, for example, “such as” or “including,” is intended merely to illustrate better the present disclosure and does not pose a limitation on the scope of the disclosure unless claimed. No language in the specification should be construed as indicating any non-claimed element as essential to the practice of the present disclosure.

### EXAMPLES

The following Examples are merely illustrative and are not intended to limit the scope or content of the disclosure in any way.

### Example 1—Identification of Privileged Nutrient Control Sequences

Functional linkage of essential gene activity to a hybrid two-component system (HTCS) activation requires identification of suitable control molecules. Characteristics of an appropriate control molecule are: safe for consumption, unable to be absorbed by the host, minimal presence in the average host diet, and unable to be consumed by host microbiota. For example, the marine polysaccharide, porphyran, found in the red algae Porphyra umbilicalis, was identified as a well-suited molecule. Additional exemplary molecules examined included agarose and anhydrotetracycline.

To identify the mobile genetic elements for polysaccharide utilization (termed a polysaccharide utilization locus or PUL), Bacteroides were diluted 200-fold into minimal media containing 200 μg/ml gentamycin and porphyran in the form of 0.8% nori extract as the sole carbon source. Selection was performed by collecting primary sewage effluent, allowing it to settle for approximately two hours and diluting it ten-fold into the media, which was then incubated anaerobically for 24 hours at 37° C. The culture was then further diluted 200-fold into the fresh media and incubated another 24 hours anaerobically at 37° C. The saturated culture was then plated as serial dilutions onto Blood-Heart-Infusion media +10% horse blood agar plates and incubated 24 hours anaerobically at 37° C. Colonies were then picked into fresh media and incubated 24 hours anaerobically at 37° C. to prepare for analysis and cryogenic storage.

Exemplary strains NB001, NB002, and NB003 were selected as capable of growth and were isolated and sequenced by Illumina MiSeq or iSeq. Homology searches were conducted to identify polysaccharide utilization loci (PULs) associated with their activity. NB001, a strain of Bacteroides ovatus, contained a PUL (SEQ ID NO: 14) having 98.1% identity to a previously published PUL for porphyran from Hehemann et al (2010), NATURE 464:908-912 and containing a putative porphyran-inducible HTCS (SEQ ID NOs: 18 and 19). A novel agarase-containing PUL was identified in NB002, a strain of Bacteroides dorei (SEQ ID NO: 15), and NB003, a strain of Bacteroides uniformis (SEQ ID NO: 16). This PUL contained a putative agarose-responsive HTCS (SEQ ID NOs: 22 and 23). NB004 demonstrated tetracycline resistance and contained a TCS-driven operon highly homologous to known tetracycline resistance genes (SEQ ID NOs: 24 and 25). The identified exemplary HTCS and TCS can be utilized to link essential gene activity to porphyran, agarose, or anhydrotetracycline.

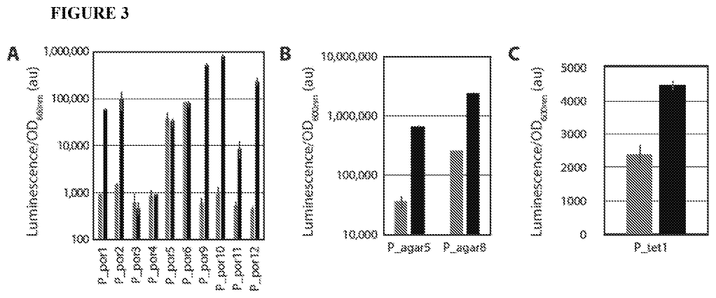

Ten candidate promoter sequences were synthesized following analysis of the >78 kilobase porphyran PUL (SEQ ID NOs: 1-10). Each candidate was coupled to a luciferase reporter gene and luminescence was quantified in the absence of porphyran or in the presence of 0.2% porphyran. Results are described in TABLE 2. Six of the promoter sequences were responsive to porphyran, with P_por10 (SEQ ID NO: 8) demonstrating the largest expression upon porphyran addition, as depicted in FIG. 3A. Additional promoters that respond to agarose (SEQ ID NOs: 22 and 23) and anhydrotetracycline (SEQ ID NOs: 24 and 25) were identified and are shown in FIG. 3B, 3C.

TABLE 2
Candidate porphyran promoters tested and porphyran-
responsive luciferase reporter assay values

| SEQ ID | Name | −Por. | +Por. | Fold Ind. |
|---|---|---|---|---|
| 1 | P_por1 | 9.2E+2 | 5.8E+4 | 63 |
| 2 | P_por2 | 1.5E+3 | 9.9E+4 | 65 |
| 3 | P_por3 | 6.3E+2 | 4.7E+2 | 1 |
| 4 | P_por4 | 8.6E+2 | 8.9E+2 | 1 |
| 5 | P_por5 | 3.8E+4 | 3.3E+4 | 1 |
| 6 | P_por6 | 8.2E+4 | 8.0E+4 | 1 |
| 7 | P_por9 | 5.8E+2 | 5.2E+5 | 894 |
| 8 | P_por10 | 9.2E+2 | 7.7E+5 | 842 |
| 9 | P_por11 | 5.4E+2 | 8.5E+3 | 16 |
| 10 | P_por12 | 4.3E+2 | 2.3E+5 | 536 |

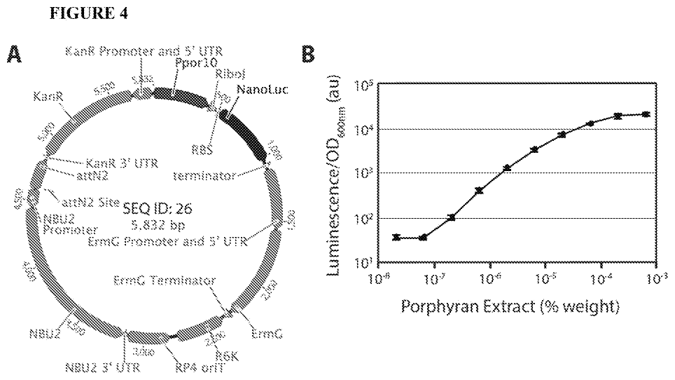

P_por10, which displayed the largest fold induction, was selected for use in biocontainment. Strain NB001 carrying a P_por10-driven luciferase, as shown in FIG. 4A, (SEQ ID NO: 26), was used to characterize the porphyran induction curve. Luciferase-protein expression was used as a reporter for porphyran-dependent transcription levels and quantified by luminescence/OD₆₀₀ₙₘ. A nearly 1,000-fold induction of luciferase was observed between concentrations of approximately 10⁻⁷ to 2×10⁻⁴ porphyran extract (weight/volume), as shown in FIG. 4B.

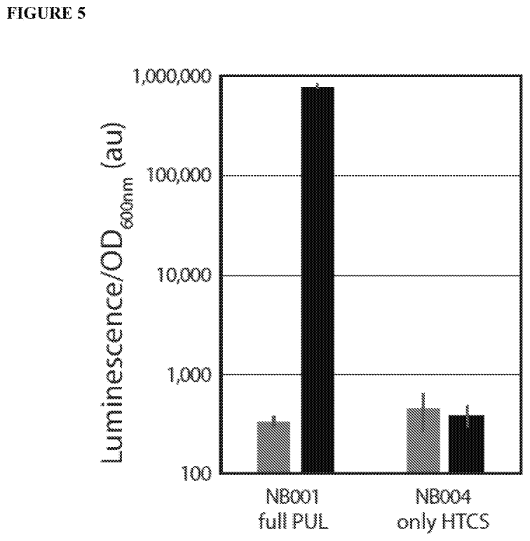

To examine if the P_por10 HTCS alone was sufficient for luciferase expression, a P_por10 luciferase construct (SEQ ID NO: 26) was altered to include expression of the porphyran HTCS (SEQ ID NOs: 18 and 19) under its native promoter. The resulting construct (SEQ ID NO: 27) was transferred to a strain either containing the full porphyran PUL, NB001, or a strain lacking the porphyran PUL, NB004. Luminescent output was measured and though the strain with the porphyran PUL demonstrated porphyran-dependent luciferase induction, the strain containing only the HTCS did not display porphyran-dependent induction (FIG. 5). These results suggest the HTCS and additional genes are required for induction of the porphyran-responsive promoters. For example, the SusC and SusD genes (SEQ ID NOs: 20 and 21), in addition to the HTCS (SEQ ID NOs: 18 and 19), may be necessary for induction of the porphyran-responsive promoters (SEQ ID NOs: 1, 2, and 7-10) on complex polysaccharides.

### Example 2—In Vitro Privileged Nutrient-Dependent Biocontainment

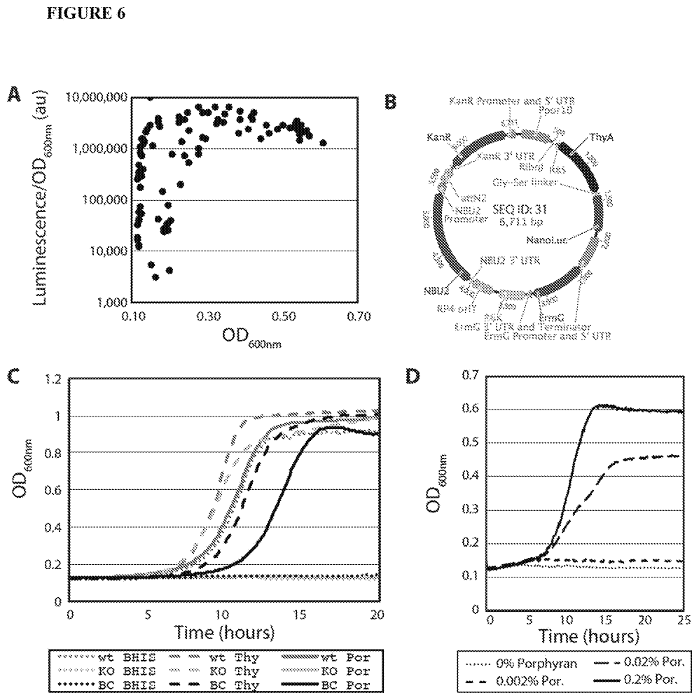

Using the PUL for porphyran growth identified in Example 1 (P_por10), a Bacteroides strain expressing porphyran-dependent induction of the essential gene thy A, thymidylate synthetase, was generated. Endogenous thy A (SEQ ID NO: 28) was knocked out using a method similar to that described in Koropatkin et al, (2008) STRUCTURE 16:1105-1115 with the modification of trimethoprim and thymidine counterselection, resulting in strain NB023. A P_por10 (SEQ ID NO: 8) driven thy A-luciferase plasmid with degenerate ribosome binding site (RBS) (SEQ ID NO: 30) was generated and is shown in FIG. 6B. The plasmid was integrated into NB023. The strain was grown in minimal media with chlorophenylalanine counterselection, streaked onto BHIS agar plates, and colonies displaying GFP positivity and/or chloramphenicol resistance were selected and validated for gene promoter replacement by PCR and Sanger Sequencing.

Individual RBS library members were assayed for thyA expression. Each was grown in media containing thymidine, then diluted into media without thymidine but containing porphyran. Strains with unique RBSs were assayed for luminescence and final OD₆₀₀ₙₘ, depicted in FIG. 6A. Strains capable of growth to high OD₆₀₀ₙₘ all displayed similar levels of luminescence, suggesting that a narrow range of thy A expression is permissible for growth. Strain NB024, which best complemented the thy A deletion, was sequenced (SEQ ID NO: 31) and selected for further experimentation.

FIG. 6C depicts the results of a growth assay for NB024, wildtype strain NB001 and thy A deletion strain NB023 in nutrient-variable media. All three strains are capable of growth in media containing thymidine (dashed lines). Only wildtype NB001 shows growth in standard BHIS media (dotted lines). In BHIS supplemented with porphyran (solid lines), NB024 grows at a level comparable to wildtype, though with a slight initial lag possibly caused by time required for thy A induction. The thy A deletion strain NB023 does not grow in BHIS media supplemented with porphyran.

Additional testing of NB024 demonstrated a porphyran-concentration dependent growth response in BHIS media depicted in FIG. 6D. Taken together, these results demonstrate functional linkage of the porphyran-responsive HTCS (SEQ ID NOs: 18 and 19) and expression of essential gene thy A.

The escape rate of NB024 biocontainment was assessed. NB024 was plated on BHIS plates supplemented with thymidine, and five individual colonies were picked. Colonies were grown at 37° C. for 14 hours in BHIS supplemented with 0.2% nori extract (porphyran). Saturated culture was then plated onto porphyran-lacking BHIS agar evenly or through serial dilutions; colonies visible after 48 hours of anaerobic growth were considered escape colonies. Approximately 1 in 3,500,00 cells displayed growth on plates lacking porphyran supplementation.

### Example 3—Engineering of Privileged Nutrient Promoter Control of Essential Native Gene in Bacteroides

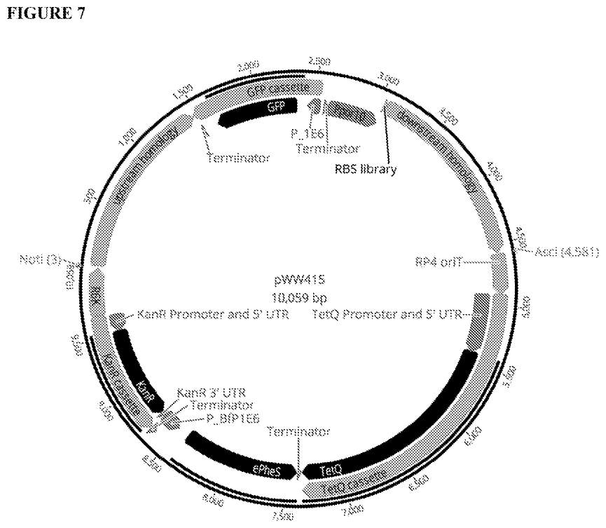

To extend the biocontainment strategy to additional essential genes, a vector was developed to replace the endogenous promoter of an essential gene with the porphyran-inducible promoter shown in FIG. 7 (SEQ ID NO: 32). This replacement method employs homologous recombination to replace the promoter of a gene of interest with a cassette containing the porphyran-inducible promoter and degenerate RBS library to find appropriate translation strength permissible for growth. Tetracycline selection allows for identification of integration of the plasmid, while counterselection on 4-chlorophenylalanine and selection of GFP positive colonies allows for identification of native promoter displacement.

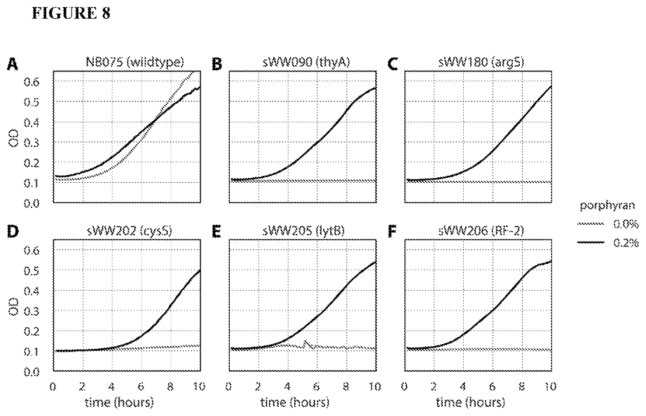

Using plasmid pWD035 (SEQ ID NO: 33), a porphyran utilization locus was integrated as described in Shepherd et al. (2018) NATURE 557:434-438 to make strain NB075. The native promoter of one of four essential genes, arginyl-tRNA synthetase (argS), cysteinyl-tRNA synthetase (cysS), penicillin tolerance protein (lytB), or peptide chain release factor (RF-2), was replaced using the promoter replacement system (SEQ ID NO: 32, 34, 35, and 36, respectively). Strains capable of growth in the presence of 0.2% porphyran were isolated and sequenced to identify appropriate translation strength. Constructs for each essential gene are as follows: argS, SEQ ID NO: 32; cysS, SEQ ID NO: 34; lytB, SEQ ID NO: 35; RF-2, SEQ ID NO: 36. Biocontained strains sWW090 (thy A), sWW180 (argS), sWW202 (cysS), sWW205 (lytB), and sWW206 (RF-2) do not grow in BHIS-only media, but do grow in BHIS-supplemented with porphyran. Results are depicted in FIG. 8.

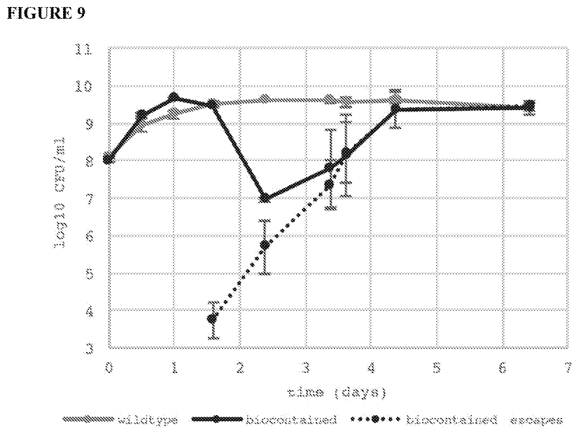

To monitor the escape dynamics and potential mechanisms of these biocontained strains, a non-biocontained and a biocontained strain were grown in a chemostat containing 0.5% porphyran, which was continuously diluted, replacing the media volume every 8.7 hours. Wildtype strain sZR0103 quickly reached and maintained a density of over 10⁹ Colony Forming Units (CFU)/ml; argS biocontained strain sZR0205 also reached a density of over 10⁹ CFU/ml but quickly dropped in optical density (about 500-fold) as the porphyran was consumed and diluted out of the media. Mutant cells of the biocontained strain that had escaped their dependence on porphyran supplementation appeared by day 2 of the assay and approached levels comparable to wildtype by day 4, as shown in FIG. 9. Sequencing of the escape strains revealed that of the 331 escape colonies evaluated, 94% of the escape colonies were one of 48 unique mutations to the HTCS that rendered it constitutively active, 4% were transposon insertions into the porphyran inducible promoter, and 2% were genomic rearrangements immediately upstream of the biocontained gene.

### Example 4—In Vitro Privileged Nutrient-Dependent Biocontainment of Bacteroides

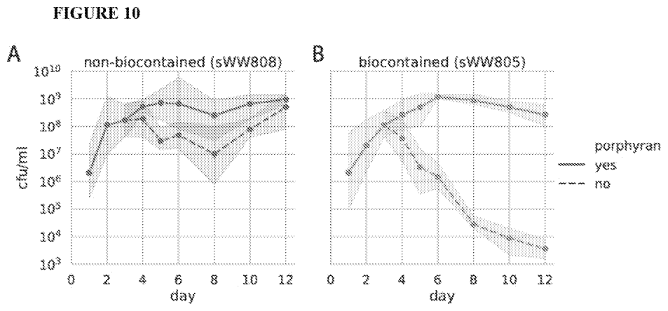

To demonstrate the efficacy of biocontainment in vivo, Sprague-Dawley rats were fed a porphyran-supplemented diet and were administered 10⁹ CFU of either sWW808, a non-biocontained strain, or sWW805, a variant of biocontained strain sWW180 carrying an additional antibiotic marker. Both strains were modified to consume porphyran, and both strains were co-administered with a non-porphyran consuming wildtype strain to ensure a competitive environment. Colonization occurred for 3-days before half the rats in each group were switched to a diet without porphyran, while the other half remained on the porphyran-supplemented diet. Strain abundance was monitored in the feces daily, and it was observed that the biocontained strain was rapidly cleared from the gut in the absence of porphyran, while the wildtype strain showed a 10-fold decrease in abundance due to the absence of its privileged nutrient, porphyran, shown in FIG. 10. When the biocontained strain was tested in a non-competitive environment, following removal of porphyran, escaping strains were found to possess mutations resulting in constitutive expression of the essential gene, similar to those characterized in Example 3.

### Example 5—Engineering of Hybrid Two Component Privileged Nutrient Control in Bacteroides

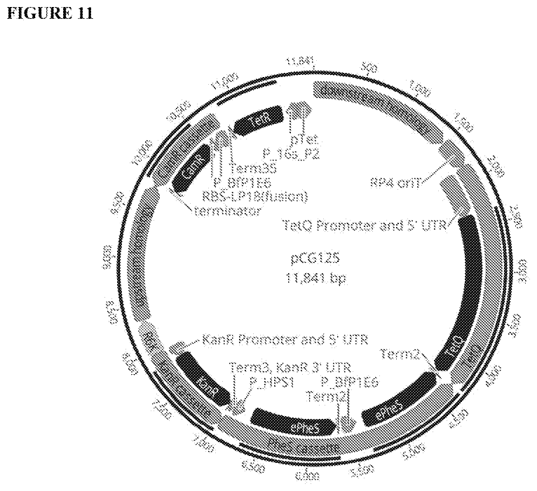

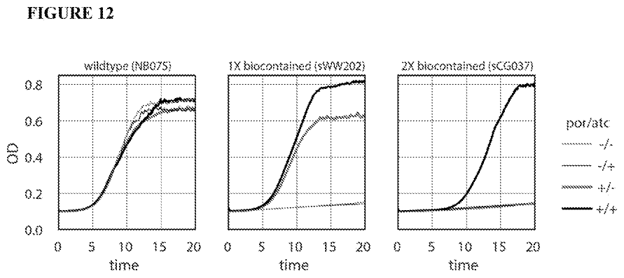

To reduce escape rates of biocontained strains, redundancy was incorporated using a second privileged nutrient control. Using the strain sWW202 with cysS expression driven by the porphyran-inducible promoter, anhydrotetracycline (aTc)-inducible control of argS expression was introduced. Incorporation of the aTc-biocontainment plasmid (SEQ ID NO: 37, FIG. 11) was performed similarly to that described in Example 3, using an aTc-inducible promoter previously described in Lim et al. (2017) CELL 169:547-558, and an RBS library to generate strain sCG037. sCG037 was predicted to require both porphyran and aTc supplementation for growth, which was observed in vitro, depicted in FIG. 12.

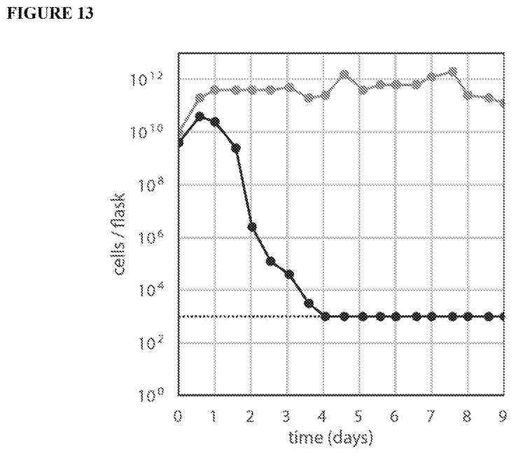

To monitor the escape dynamics and to assess if redundancy reduces escape rate, a non-biocontained strain (NB075) and double-biocontained strain sCG037 were grown in a chemostat containing 0.2% porphyran and 10 ng/ml aTc, which were serially diluted out of the media. Both strains initially reached a density of over 10⁹ CFU, which decreased upon removal of the porphyran and aTc from the media to the limit of detection (10^{3.5} cells/flask) by day 4. At day 7, porphyran and aTc were added back to the media in order to assess if any biocontained cells had survived and were capable of growth. No growth of the biocontained strain was detected after 2 days, suggesting all double-biocontained cells had been cleared. Results are depicted in FIG. 13.

### Example 6—Engineering of Chimeric Hybrid Two Component Privileged Nutrient Control in Bacteroides

To simplify therapeutic strains such that administration of a single control molecule is linked to expression of multiple essential genes, chimeric HTCSs were designed. In one embodiment of such a chimeric HTCS, the sensor of one HTCS is linked to the DNA-binding region of a second HTCS. This can be done by replacing the sensor domain of the second HTCS with the sensor domain of the first HTCS such that the chimeric HTCS senses the control molecule of the first HTCS but targets a different promoter than the first HTCS.

HTCSs with a signal transduction Y_Y_Y domain, with high homology to the porphyran Y_Y_Y domain (SEQ ID NO: 19, residues 683-747) were examined for use in the generation of chimeric HTCSs. As it is important to consider that the newly designed promoter only responds to the chimeric HTCS and not to molecules produced by or commonly encountered by the host or to other HTCSs or other regulators native to the host, the HTCS should contain regulatory domains either absent or rarely found in the biocontained strain. Accordingly, the set was refined by removing HTCSs with high homology to other HTCS regulatory domains, particularly those in the target strain.

A first HTCS from Bacteroides nordii (SEQ ID NO: 51), a second HTCS from Bacteroides nordii (SEQ ID NO: 38), and an HTCS from Bacteroides salyersiae (SEQ ID NO: 52) were selected for experimentation. The C-terminal region (containing the regulatory domain) of each of these three HTCSs was fused to the N-terminal region (containing the porphyran-sensor domain) of the porphyran HTCS (SEQ ID NO: 19, as described in Example 1). We tested a number of different fusion locations, and found that the location immediately downstream of the Y_Y_Y domain of the porphyran HTCS, within 5 residues of the putative periplasmic side of the inner membrane (residue 753 in the porphyran HTCS, SEQ ID NO: 19), was the most reliable location for generating functional chimeras. A chimeric HTCS was generated including the sensor domain of the porphyran HTCS and the regulatory domain of the first HTCS from Bacteroides nordii. This HTCS is referred to HTCS-17106 (SEQ ID NO: 53) and an exemplary vector encoding HTCS-17106 is referred to as pWW1266 (SEQ ID NO: 55). A chimeric HTCS was generated including the sensor domain of the porphyran HTCS and the regulatory domain of the HTCS from Bacteroides salyersiae. This HTCS is referred to as HTCS-10809 (SEQ ID NO: 54) and an exemplary vector encoding HTCS-10809 is referred to as pWW1265 (SEQ ID NO: 56). A chimeric HTCS was generated including the sensor domain of the porphyran HTCS and the regulatory domain of the second HTCS from Bacteroides nordii. This HTCS is referred to as HTCS-17150 (SEQ ID NO: 39) and an exemplary vector encoding HTCS-17150 is referred to as pWW1267 (SEQ ID NO: 40). A schematic of pWW1267 is shown in FIG. 14B.

Promoters responsive to each of the chimeric HTCSs were identified. A promoter responsive to HTCS-17106 is depicted in SEQ ID NO: 62, and a promoter responsive to HTCS-10809 is depicted in SEQ ID NO: 63. Luciferase reporters for each of the chimeric HTCSs were generated by coupling the corresponding promoter to a luciferase gene. The luciferase reporter for HTCS-17106 is depicted in SEQ ID NO: 57, the luciferase reporter for HTCS-10809 is depicted in SEQ ID NO: 58, and the luciferase reporter for HTCS-17150 is depicted in SEQ ID NO: 41. Bacteroides vulgatus strains containing a porphyran utilization locus (as described in Example 3) and one of the luciferase reporters above were further modified with either an empty vector or a construct that expressed the associated chimeric HTCS. In the presence of the chimeric HTCS, porphyran-responsive luciferase expression was observed for each chimeric HTCS, as shown in FIG. 14C. The chimeric HTCSs can, for example, be used in combination with the wildtype porphyran-responsive HTCS in order to reduce biocontainment escape rates, similarly to the system described in Example 5, with the advantage of using a single control molecule.

### Example 7—Engineering Improved Chimeric Hybrid Two Component Systems via Targeted Mutation

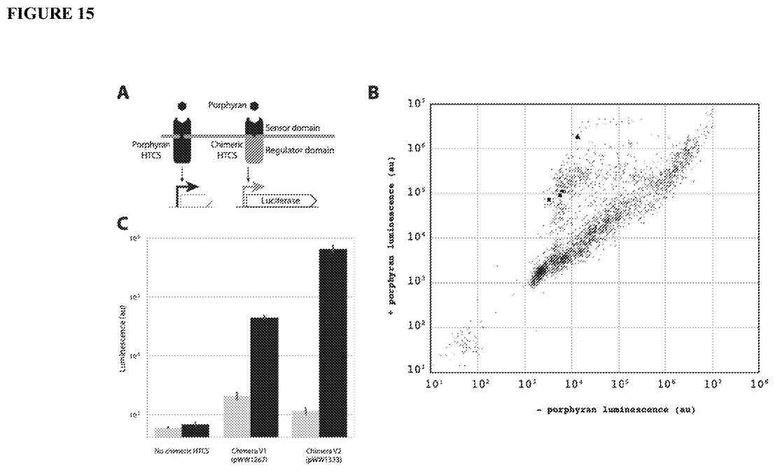

To aid in the generation of biocontained strains, HTCS-17150 (SEQ ID NO: 39, as described in Example 6) was mutated to improve porphyran responsiveness. Residues in the transmembrane region (residues 753 through 777) were targeted for mutation by amplification with degenerate oligos, and the resulting variants of the pWW1267 (SEQ ID NO: 40) expression construct were added to Bacteroides vulgatus strains containing a porphyran utilization locus (as described in Example 3) and the chimeric HTCS-associated luciferase reporter (SEQ ID NO: 41, as described in Example 6), as shown in FIG. 15A. Strains including the HTCS-17150 mutants were then screened for activity in the presence or absence of porphyran. Results are shown in FIG. 15B. Each point in FIG. 15B represents a strain expressing an HTCS-17150 mutant, with points along the diagonal no longer responding to porphyran and points in the upper left portion of the plot showing the desired higher activity in the presence of porphyran and lower activity in the absence of porphyran. Compared to the control (strains expressing the unmutated HTCS-17150, shown as squares in FIG. 15B), a number of strains were identified with improved porphyran responsiveness. Select strains were restreaked and tested in replicate, as shown in FIG. 15C. An exemplary strain including the construct pWW1333 (SEQ ID NO: 60) showed lower activity in the absence of porphyran and higher activity in the presence of porphyran. pWW1333 expressed a mutant HTCS-17150 referred to as HTCS-17150v2 and having an amino acid sequence shown in SEQ ID NO: 59. Additional improved mutant HTCSs referred to as HTCS-17150v3-HTCS-17150v10 have amino acid sequences shown in SEQ ID NOs: 64-71, respectively.

### Example 8—Orthogonality of Engineered Chimeric Hybrid Two Component Systems

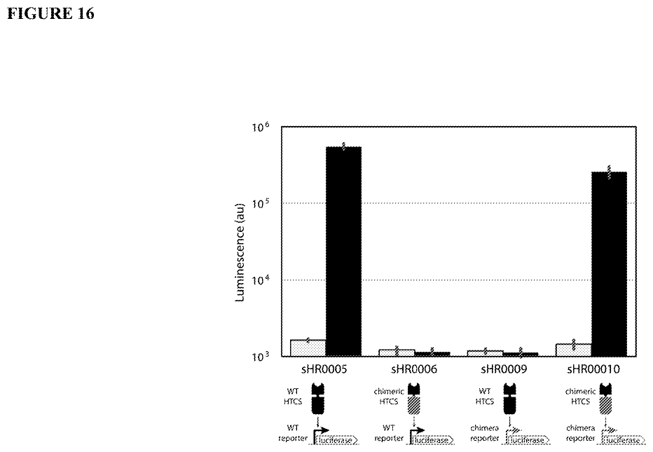

When a first and a second HTCS (e.g., a wildtype HTCS and a chimeric HTCS) are used to implement double-biocontainment, it is important that activation of the first HTCS does not activate the promoter associated with the second HTCS. Otherwise, an activating escape mutation in a single HTCS could be sufficient for escape. To demonstrate orthogonality of the HTCSs described in this Example, we tested (i) the wildtype porphyran-responsive HTCS (SEQ ID NO: 19) in combination with a HTCS-17150v2-responsive promoter (SEQ ID NO: 45), and (i) the chimeric HTCS-17150v2 (as described in Example 7) in combination with a wildtype porphyran-responsive promoter (SEQ ID NO: 8). Each HTCS was also tested with its associated promoter as a control. The results are shown in FIG. 16, and show that the promoters associated with the wildtype porphyran-responsive HTCS and HTCS-17150v2 are not activated in the presence of the other HTCS, and only activated when the associated HTCS and porphyran are both present.

### Example 9—Engineering Double Hybrid Two Component System Privileged Nutrient Control in Bacteroides

This Example describes the generation of strains including a first and a second HTCS (a porphyran-responsive wildtype HTCS and a porphyran-responsive chimeric HTCS) to implement double-biocontainment.

A Bacteroides vulgatus strain (sWW810) was modified to be capable of porphyran consumption (using plasmid pWD035 (SEQ ID NO: 33) as described in Example 3) and also express a chimeric HTCS (SEQ ID NO: 59, as described in Example 7). The strain was further modified to replace the native promoter of the essential gene penicillin tolerance protein (lytB) with a promoter responsive to the HTCS (SEQ ID NO: 45). The promoter was replaced using the promoter replacement system described above in Example 3. Briefly, this replacement method employs homologous recombination to replace the native promoter with a cassette containing the promoter of interest and degenerate RBS library to find the appropriate translation strength permissible for growth. A biocontained strain capable of growth only in the presence of 0.2% porphyran was isolated, and is referred to as sWW939. A construct including the cassette from sWW939, with the appropriate resulting translation strength, is referred to as pZR3007 (SEQ ID NO: 61).

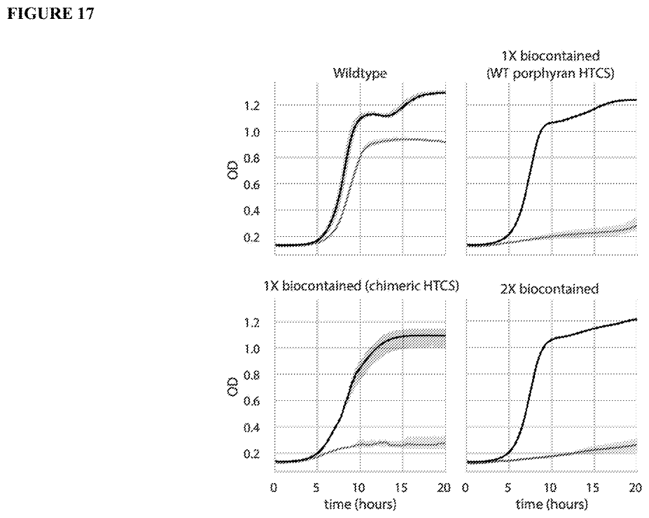

Strain sWW180 (as described in Example 3, and biocontained with the wildtype porphyran HTCS driving expression of argS) was further modified with pZR3007 to produce a double biocontained strain (sWW942) that also had lytB under control of the chimeric HTCS. The non-biocontained (NB075), the two single biocontained strains (sWW180) and sWW939) and the double biocontained strain (sWW942), were tested for growth in BHIS media only and BHIS media supplemented with porphyran. Results are shown in FIG. 17.

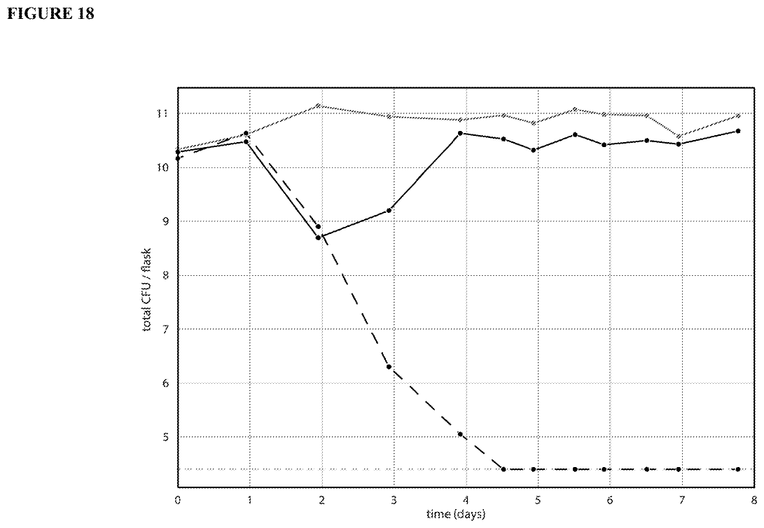

To compare growth dynamics and potential escape ability, the non-biocontained (NB075), the single biocontained strains (sWW180), and the double biocontained strain (sWW942) were grown in a chemostat initially containing 0.5% porphyran, which was continuously diluted with media lacking porphyran, replacing the media volume every 11 hours (similar to the experimental setup associated with FIG. 9). Results are shown in FIG. 18. The non-biocontained strain (NB075) quickly reached and maintained a density of over 10⁹ CFU/ml. The single biocontained strain (sWW180) also reached a density of over 10⁹ CFU/ml but initially quickly dropped in density (more than 100-fold) as the porphyran was consumed and diluted out of the media. However, the single biocontained strain approached levels comparable to wildtype by day 4, as mutant cells of the biocontained strain escaped their dependence on porphyran supplementation. The double biocontained strain (sWW942) initially dropped in density similarly to the single biocontained strain, but escape mutants never appeared and the density dropped to below the limit of detection. After 32 day's, porphyran was added to the media to encourage outgrowth of any surviving double biocontained cells, but after three days on porphyran no cells could be recovered from the double biocontained chemostat. This indicates that the chemostat that at one point harbored more than 30 billion cells had been sterilized by double biocontainment in rich media lacking porphyran.

### Example 10—In Vivo Biocontainment in Mice Harboring Human Microbiota

This Example describes biocontainment in vivo in mice that harbor a human microbiota.

A Bacteroides vulgatus strain was modified to be capable of porphyran consumption (using plasmid pWD035 (SEQ ID NO: 33)) to produce strain NB144. NB144 was further modified for biocontainment using plasmid pZR2837 (SEQ ID NO: 72) to produce strain sZR0323. In strain sZR0323, argS is associated with a RBS (SEQ ID NO: 47), and under control of a promoter (SEQ ID NO: 73) that is responsive to a porphyran HTCS (SEQ ID NO: 19).

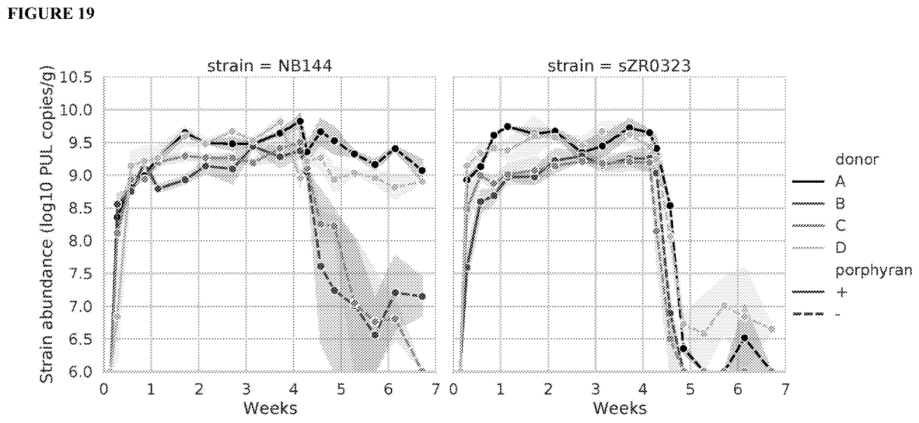

Germ free Swiss-Webster mice were colonized with microbiota from one of four anonymous healthy human donors (donors A-D). After 3 weeks of microbiota stabilization, mice were administered 10⁹ CFU of either NB144 or sZR0323 and fed a porphyran-supplemented diet. Strain abundance was monitored in the feces daily via quantitative polymerase chain reaction (QPCR) to quantify the number of copies of the porphyran utilization locus. Results are shown in FIG. 19. Both strains reached a colonization level of at least 10⁹ cells/g feces within the first week, and remained between 10⁹ and 10¹⁰ cells/g for the period in which porphyran was included in the diet. After 4 weeks porphyran was removed from the diet. After the diet switch, in the groups of mice containing microbiotas from donors B and C, it was observed that both the non-biocontained and the biocontained strain dropped substantially in abundance, with the non-biocontained strain dropping more than 100-fold and the biocontained strain dropping even further to below the limit of detection of 10⁶ cells/g feces. In the other groups of mice, containing microbiotas from donors A and D, it was observed that the non-biocontained strain remained at a high abundance of about 10⁹ cells/g feces, but the biocontained strain dropped about 1000-fold in abundance. This data shows that the biocontained strain is substantially attenuated in the context of mice harboring human microbiota.

### Example 11—Engineering of Complementary Biocontainment Mechanisms with Privileged Nutrient Control in Bacteroides

The biocontainment strategies described in previous Examples can be further modified by the addition of complementary biocontainment mechanisms. One such mechanism is the establishment of a competitive ecosystem through introduction of a non-engineered, competing strain lacking the ability to grow on porphyran but retaining all other polysaccharide utilization capabilities. Another such mechanism is through deletion of genes in the biocontained strain that significantly impairs the fitness of the strain when not grown in the presence of porphyran, such as a polysaccharide utilization locus involved in polysaccharide metabolism.

### INCORPORATION BY REFERENCE

The entire disclosure of each of the patent and scientific documents referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting on the disclosure described herein. Scope of the disclosure is thus indicated by the appended claims rather than by the foregoing description, and all changes that come within the meaning and range of equivalency of the claims are intended to be embraced therein.

1. A genetically modified bacterium comprising:
(a) a first activator that is activated by a control molecule;
(b) a first promoter that is activated by the first activator; and
(c) a first essential gene that is operably linked to the first promoter, and optionally:
(d) a second activator that is activated by the control molecule;
(e) a second promoter that is activated by the second activator; and
(f) a second essential gene that is operably linked to the second promoter.

2. The bacterium of claim 1, wherein the first promoter is not activated by the second activator and the second promoter is not activated by the first activator.

3. The bacterium of claim 1, further comprising:
(g) a third activator that is activated by the control molecule;
(h) a third promoter that is activated by the third activator; and
(i) a third essential gene that is operably linked to the third promoter.

4. The bacterium of claim 3, wherein the third promoter is not activated by the first or second activator and the third promoter is not activated by the first or second activator.

5. The bacterium of claim 1, wherein the expression of the first and/or second essential gene is dependent upon the presence of the control molecule.

6-8. (canceled)

9. The bacterium of claim 1, wherein the control molecule is selected from a marine polysaccharide and an antibiotic or a derivative thereof.

10. The bacterium of claim 9, wherein the marine polysaccharide is selected from a porphyran and agarose.

11. (canceled)

12. The bacterium of claim 1, wherein the first and/or second activator is a two-component system (TCS) protein comprising a sensor domain and a regulatory domain.

13. The bacterium of claim 1, wherein the first and/or second activator is a hybrid two-component system (HTCS) protein comprising a sensor domain and a regulatory domain.

14. (canceled)

15. The bacterium of claim 13, wherein the HTCS protein is a chimeric HTCS protein, wherein the sensor domain is a sensor domain from a first naturally-occurring HTCS protein, or a functional fragment or variant thereof, and the regulatory domain is a regulatory domain from a second naturally-occurring HTCS protein, or a functional fragment or variant thereof.

16-18. (canceled)

19. The bacterium of claim 13, wherein the HTCS protein comprises an amino acid sequence having at least 80% identity to any one of SEQ ID NOs: 19, 23, 25, 38, 39, 42, 43, 51, 52, 53, 54, 59, or 64-71, or a functional fragment or variant thereof.

20. The bacterium of claim 1, wherein the bacterium comprises one or more transgenes encoding the first and/or second activator.

21. The bacterium of claim 1, wherein the first and/or second promoter comprises a nucleotide sequence having at least 80% identity to any one of SEQ ID NOs: 1, 2, 7, 8, 9, 10, 11, 12, 13, 45, 46, 62, 63, or 73, or a functional fragment or variant thereof.

22. The bacterium of claim 21, wherein the essential gene is selected from thymidylate synthase (ThyA), arginyl-tRNA synthetase (argS), cysteinyl-tRNA synthetase (cysS), penicillin tolerance protein (lytB) and peptide chain release factor (RF-2).

23. The bacterium of claim 1, wherein the first and/or second activator and/or promoter is heterologous to the bacterium.

24. (canceled)

25. The bacterium of claim 1, wherein culturing of the bacterium results in a bacterium that is capable of growth and/or viability in the absence of the control molecule at a frequency of less than 10⁻⁵, 10⁻⁶, 10⁻⁷, 10⁻⁸, or 10⁻⁹.

26. The bacterium of claim 1, wherein, following culture of the bacterium with the control molecule and subsequent removal of the control molecule from the culture, the half-life of the bacteria in culture is less than a day.

27. The bacterium of claim 1, wherein, following administration of the bacterium and control molecule to a subject, the amount of bacteria in the subject decreases 10 fold within 2 days of removal or discontinuation of the control molecule from the subject.

28. The bacterium of claim 1, wherein the control molecule is a porphyran and the first and second activator are each an HTCS protein, and
(i) the porphyran, when present, activates the first and second HTCS proteins,
(ii) the first and second HTCS proteins, when activated, activate the first and second promoters, respectively, and
(iii) the first and second promoters, when activated, direct expression of the first and second essential genes, respectively, thereby resulting in the growth and/or viability of the bacterium being dependent upon the presence of the porphyran.

29-31. (canceled)

32. The bacterium of claim 1, further comprising one or more transgenes encoding a protein, or a functional fragment or variant thereof, selected from SusC and SusD.

33. The bacterium of claim 1, wherein the bacterium comprises one or more transgenes that increase its ability to utilize a privileged nutrient as carbon source.

34. The bacterium of claim 33, wherein the privileged nutrient is a marine polysaccharide.

35. The bacterium of claim 34, wherein the marine polysaccharide is porphyran.

36. The bacterium of claim 1, further comprising one or more therapeutic transgenes.

37-39. (canceled)

40. The bacterium of claim 36, wherein the therapeutic transgene is operably linked to a promoter, and the promoter comprises the consensus sequence GTTAA(n)₄₋₇GTTAA(n)₃₄₋₃₈TA(n)₂TTTG.

41. The bacterium of claim 36, wherein the therapeutic transgene is operably linked to a promoter, and the promoter comprises SEQ ID NO: 48, SEQ ID NO: 49, or SEQ ID NO: 50.

42. (canceled)

43. A pharmaceutical composition comprising the bacterium of claim 1 and a pharmaceutically acceptable excipient.

44-46. (canceled)

47. A method for reducing the growth and/or viability of a bacterium in the absence of a control molecule, the method comprising genetically modifying the bacterium to comprise:
(a) a first activator that is activated by the control molecule;
(b) a first promoter that is activated by the first activator; and
(c) a first essential gene that is operably linked to the first promoter.

48. The method of claim 47, further comprising genetically modifying the bacterium to comprise:
(d) a second activator that is activated by the control molecule;
(e) a second promoter that is activated by the second activator; and
(f) a second essential gene that is operably linked to the second promoter.

49. The method of claim 48, further comprising genetically modifying the bacterium to comprise:
(g) a third activator that is activated by the control molecule;
(h) a third promoter that is activated by the third activator; and
(i) a third essential gene that is operably linked to the third promoter.

50. A method of colonizing the gut of a subject, the method comprising administering the bacterium of claim 1 to the subject.

51. A method of treating a disease or disorder in a subject in need thereof, the method comprising administering the bacterium of claim 1 to the subject.

52-57. (canceled)
